SEQUENCE LISTING (1 sequence)

SEQ ID NO: 1 - RNA, 25 nt, Artificial Sequence
  Guide RNA for knock-out of SLC16A1
caccgacagacgtatagttgctgta

(19) United States
(12) Patent Application Publication
(10) Pub. No.: US 2024/0270749 A1
(43) Pub. Date: Aug. 15, 2024

(54) 1,2,4-TRIAZOLO[1,5-A]PYRIMIDINE-BASED SLC16A3 INHIBITORS AND THEIR THERAPEUTIC USE

(71) Applicant: CEMM - FORSCHUNGSZENTRUM FÜR MOLEKULARE MEDIZIN GMBH, Wien (AT)

(72) Inventors: Vojtech DVORAK, Wien (AT); Andrea CASIRAGHI, Wien (AT); Giulio SUPERTI-FURGA, Wien (AT)

(73) Assignee: CEMM - FORSCHUNGSZENTRUM FÜR MOLEKULARE MEDIZIN GMBH, Wien (AT)

(21) Appl. No.: 18/562,029

(22) PCT Filed: May 23, 2022
(86) PCT No.: PCT/EP2022/063943

§ 371 Date: Nov. 17, 2023

(30) Foreign Application Priority Data
May 21, 2021 (EP) 21175419.7

(51) Int. Cl. C07D 487/04; A61K 31/519; A61K 31/55; A61P 35/00

(52) U.S. Cl. CPC C07D 487/04; A61K 31/519; A61K 31/55; A61P 35/00

(57) ABSTRACT

The present invention provides 1,2,4-triazolo[1,5-a]pyrimidine-based compounds of formula (I), Formula (I) and pharmaceutically acceptable salts thereof, which have been found to be advantageously potent and selective inhibitors of SLC16A3, as well as their use in the treatment or prevention of SLC16A3-associated diseases/disorders such as cancer.

The present invention provides 1,2,4-triazolo[1,5-a]pyrimidine-based compounds of formula (I) and pharmaceutically acceptable salts thereof, which have been found to be advantageously potent and selective inhibitors of SLC16A3, as well as their use in the treatment or prevention of SLC16A3-associated diseases/disorders such as cancer.

Lactate, the end product of glycolysis, is probably best known as a waste product of cellular metabolism. However, lactate can be also used as an energy source. In fact, it has been showed that, in many tissues, highly glycolytic cells are secreting lactate which is then used as an energy source by neighboring cells (Draoui N et al., DMM Disease Models and Mechanisms, 2011, 4(6):727-32). This metabolic cooperation was for example described in skeletal muscles, brain, testis or tumor microenvironment (TME).

The Warburg effect, which is characteristic for many tumors, is manifested by glycolysis even in aerobic conditions. The metabolism of many cancer cells is connected to a massive increase in glucose consumption and secretion of lactate, allowing for regeneration of NAD⁺ which is required to sustain cellular processes. Elevated lactate levels play many roles in the TME, contributing to many aspects of malignancy, such as stimulation of angiogenesis, cancer cell immune evasion or resistance to therapy (Parks S K et al., Annual Review of Cancer Biology, 2020, 4:141-58).

Lactate is transported through the plasma membrane mainly by monocarboxylate transporters (MCTs) of the SLC16 family (Rabinowitz J D et al., Nature Metabolism, 2020, 2(7):566-71). The SLC16 family consists of 14 members, out of which 4 are capable of transporting lactate and pyruvate—i.e., SLC16A1, SLC16A3, SLC16A7 and SLC16A8 (Bosshart P D et al., Trends in Biochemical Sciences, 2020, 1-13). From these, mainly SLC16A1 (MCT1) and SLC16A3 (MCT4) have been studied in cancer, particularly in context of metabolic symbiosis (Lyssiotis C A et al., Trends in Cell Biology, 2017, 27(11):863-75).

Metabolic symbiosis is established as a response to a rapid growth of tumor and the resulting creation of zones with different oxygen availability. Tumor cells in hypoxic regions metabolize glucose through anaerobic glycolysis, resulting in rapid production of lactate, which is then released to the microenvironment through SLC16A3 (expression of SLC16A3 is upregulated by HIF-1α). Lactate is then imported to tumor (or stromal) cells in tumor regions with higher oxygen availability through SLC16A1, where it serves as a fuel for mitochondrial metabolism. The role that both SLCs are playing in the metabolic symbiosis is to some extent dictated by affinities of both transporters for lactate and pyruvate. While SLC16A1 has a high affinity for pyruvate, the pyruvate affinity of SLC16A3 is low, facilitating a pyruvate conversion into lactate in SLC16A3 expressing cells, which is connected to regeneration of NAD⁺. At the same time, it was shown that SLC16A3 is responsible for lactate export even in microenvironment with high lactate concentration, supporting a role of SLC16A3 in lactate export. Metabolic symbiosis has been reported in pancreatic neuroendocrine cancer, pancreatic ductal adenocarcinoma, lung cancer, breast cancer and colon cancer, suggesting that this may be a general phenomenon in many cancer types and thus an interesting therapeutic target.

While selective and specific inhibitors for SLC16A1 exist—namely AZD3965 and BAY-8002—and AZD3965 recently underwent clinical trials for the treatment of advanced solid tumors or lymphomas (NCT01791595), selective inhibitors of SLC16A3 are still lacking completely (Quanz M et al., Molecular Cancer Therapeutics, 2018, 17(11):2285-96). Until recently, the best available inhibitor of SLC16A3 was syrosingopine, which inhibits both SLC16A1 and SLC16A3 with higher selectivity toward SLC16A3 (Benjamin D et al., Cell Reports, 2018, 25(11):3047-3058.e4). Since inhibition of SLC16A1 is also affecting a T cell function (Murray C M et al., Nature Chemical Biology, 2005, 1(7):371-76), targeting SLC16A3 may in some cancers disrupt metabolic symbiosis while preserving functions of immune cells and is thus considered to be a superior target compared to SLC16A1. Recently, a novel potent and selective inhibitor of SLC16A3, i.e. VB124, was introduced (Cluntun A A et al., Cell Metabolism, 2020, 1-20).

SLC16A3 is frequently upregulated in tumors, compared to normal state, higher expression in many cases correlates with worse prognosis, and in some cases even elevated expression of SLC16A3 was found in metastatic lesions compared to primary tumors.

SLC16A3 was reported to play a role in breast cancer (Baenke F et al., Journal of Pathology, 2015, 237(2):152-65). The same study found that expression of SLC16A3 is regulated by PI3K-Akt signaling axis and that the sensitivity to SLC16A3 perturbation was strongest in HER2⁺ subset of cell lines and in cell lines with high levels of Akt phosphorylation. At the same time, breast cancer cell lines increased glutamine consumption and became more sensitive to inhibition of glutaminase upon SLC16A3 silencing. Moreover, SLC16A3 expression is elevated in invasive breast cancer and high expression of SLC16A3 correlates with poor prognosis in breast cancer patients (Baenke F et al., Journal of Pathology, 2015, 237(2):152-65; Doyen J et al., Biochemical and Biophysical Research Communications, 2014, 451(1):54-61).

A recent study showed that basigin (CD147) is frequently demethylated in clinical samples of non-small lung cancer (NSCLC), which leads to increase in interaction with SLC16A3, resulting in higher lactate export (the study did not investigated effects on SLC16A1) (Wang K et al., Cell Metabolism, 2021, 33(1):160-173.e6). Moreover, the demethylated form of basigin correlated with N staging, pathological grading and high expression correlated with poor overall survival. Another study focusing on NSCLC mouse xenograft model found that development resistance to tyrosine kinase inhibitors (EGFR and MET) is caused by elevated lactate in TME (Apicella M et al., Cell Metabolism, 2018, 28(6):848-865.e6). This effect was diminished by silencing of SLC16A3 in tumor cells, resulting in re-sensitization of tumor cells to therapy.

Similarly, SLC16A3 was shown as a therapeutic target for tumors resistant to therapy targeting tumor angiogenesis. In mouse models of pancreatic neuroendocrine cancer, breast cancer and renal cancer, resistance to angiogenesis inhibitors develops through metabolic compartmentalization and development of metabolic symbiosis (Allen E et al., Cell Reports, 2016, 15(6):1144-60; Jimenez-Valerio G et al., Cell Reports, 2016, 15(6):1134-43; Pisarsky L et al., Cell Reports, 2016, 15(6):1161-74). These results were confirmed also in clinical samples from renal cancer (Jiménez-Valerio G et al., Cell Reports, 2016, 15(6):1134-43). Silencing of SLC16A3 resulted in disruption of metabolic symbiosis and re-sensitization of tumors to angiogenesis targeting therapy.

SLC16A3 is one of the most overexpressed genes in renal cancer, and was found to be an essential gene for renal cancer cell lines in genome-wide siRNA screen (Gerlinger M et al. Journal of Pathology, 2012, 227(2):146-56). The study also assessed SLC16A3 expression in clear cell renal carcinoma patient biopsies by immunohistochemistry and found that SLC16A3 was detectable in a majority of samples, and that expression correlates with Fuhrman nuclear grade. Moreover, metastatic lesions had significantly higher SLC16A3 mRNA levels compared to primary tumors.

SLC16A3 was also shown to play a role in bladder cancer. Expression of SLC16A3 was elevated in primary tumors compared to benign urothelium and high SLC16A3 expression associates with inferior overall survival (Todenhofer T et al., Molecular Cancer Therapeutics, 2018, 17(12):2746-55). Silencing of SLC16A3 reduced growth of bladder cancer both in vitro and in orthotopic xenograft mouse model.

A study focusing on the role of SLC16A3 in pancreatic cancer showed that expression of SLC16A3 is elevated in tumors compared to normal tissue and elevated expression of SLC16A3 correlates with worse prognosis (no matter if high SLC16A3 expression is found in the tumor stroma or cancer cells) (Baek G H et al., Cell Reports, 2014, 9(6):2233-49). The study found that silencing of SLC16A3 in pancreatic cell lines results in metabolic crisis, which can be compensated by upregulation of mitochondrial metabolism, switch from glucose to glutamine as an energy source, or by induction of macropinocytosis or autophagy. Furthermore, silencing of SLC16A3 resulted in cell death in cell lines with high endogenous expression of SLC16A3. Moreover, inhibition of mitochondrial complex 1 (phenformin, rotenone), macropinocytosis (EIPA), or autophagy (chloroquine) had synergistic effect with silencing of SLC16A3 in cell lines with high endogenous expression of SLC16A3. Silencing of SLC16A3 also showed significantly slower tumor growth in mouse xenografts.

The role of lactate transporters from the SLC16 family was also investigated in colorectal cancer. One study using cell lines, mouse xenografts and patient samples from primary tumors and peritoneal metastasis found that silencing of SLC16A1 and SLC16A3 (but not SLC16A7) reduce tumor growth in xenograft model (Kim H K et al., Molecular Cancer Therapeutics, 2018, 17(4):838-48). Silencing of both transporters showed additive effect with radiotherapy or treatment with 5-fluorouracil. Importantly, cells obtained from malignant ascites metastasis of colorectal cancer patients showed high overexpression of SLC16A3 and were sensitive to silencing of SLC16A3. The study also showed that patients with SLC16A3 expression showed significantly shorter recurrence-free survival after curative surgery compare to patients with no or low expression of SLC16A3 in their tumors.

SLC16A3 has been implicated not only in cancer but also in other diseases. A recent study showed that SLC16A3 is a therapeutic target for heart failure and the use of a new selective and potent SLC16A3 inhibitor, VB-124, can prevent cardiac hypertrophy (both in vitro and in a mouse model) caused by disbalance of the pyruvate-lactate axis (Cluntun A A et al., Cell Metabolism, 2020, 1-20; Cluntun A A et al., Cell Metabolism, 2021, 33(3):629-648.e10).

Patients with rheumatoid arthritis (RA) have frequently low pH of synovial fluid with increased levels of lactate. One study focusing on investigating the role of transporters involved in regulation of pH (SLC9A1, V-ATPase, SLC16A1 and SLC16A3) found elevated expression of SLC16A3 in synovial fibroblast of RA patients (RASFs) compared to synovial fibroblasts of osteoarthritis patients (OASFs) (Fujii W et al., Arthritis and Rheumatology, 2015, 67(11):2888-96). In vitro studies showed that silencing of SLC16A3 induced apoptosis of RASFs, but not OASFs. This finding was then confirmed in a mouse model of collagen-induced arthritis by in vivo electroporation of SLC16A3-targeting siRNA, resulting in reduction of synovial cell hyperplasia and infiltration of inflammatory cells. These results suggest that SLC16A3 is a potential therapeutic target for RA.

SLC16A3 has further been described as a potential target for the therapy of inflammatory bowel disease (He L et al., Dis Markers, 2018, 2018:2649491; Zhang S et al., Cell Prolif, 2019, 52(6):e12673) and Alzheimer's disease (Hong P et al., Neurotoxicology, 2020, 76:191-199).

SLC16A3 inhibitors and therapeutic uses thereof have been proposed in WO 2017/196936, WO 2018/111904, WO 2019/215316 and US 2019/0352282. Moreover, WO 2016/081464 relates to inhibitors of the MCT (SLC16) family in general, without addressing the crucial question of specificity for SLC16A3. Recently, two papers using the SLC16A3 inhibitor VB124 were published (Bonglack E N et al., BioRxiv, 2020, 12.04.410563; Cluntun A A et al., Cell Metabolism, 2020, 1-20). AstraZeneca presented data on their SLC16A3 inhibitor AZD0095 at the AACR Annual Meeting 2019 in Atlanta (Critchlow S E et al., Cancer Res, 2019, 79(13 Suppl): Abstract 1207, doi:10.1158/1538-7445.AM2019-1207).

WO 2017/112777 does not relate to SLC16A3 inhibitors but describes certain compounds having a triazolopyrimidine scaffold as inhibitors of MAPK1/2 phosphorylation.

To date, in spite of all previous and ongoing efforts to develop selective inhibitors of SLC16A3, no such compounds have obtained regulatory approval. Thus, there is still an urgent and unmet need for novel and improved of inhibitors SLC16A3, particularly for potent and selective SLC16A3 inhibitors.

The present invention addresses this need and provides compounds that have been found to exhibit a highly potent and selective inhibitory effect on SLC16A3, which renders these compounds particularly advantageous for therapeutic use, including in the treatment or prevention of cancer and other SLC16A3-associated diseases/disorders. The invention thus solves the problem of providing improved SLC16A3 inhibitors.

Accordingly, the present invention provides a compound of the following formula (I) or a pharmaceutically acceptable salt thereof:

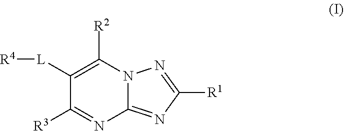

In formula (I), the group R¹ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

R² and R³ are each independently selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

R⁴ is aryl or heteroaryl, wherein said aryl or said heteroaryl is optionally substituted with one or more groups R⁴¹, and further wherein said heteroaryl is not 1,2,4-triazolyl.

Each R⁴¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

L is C₂₋₈ alkylene, wherein one —CH₂— unit in said alkylene is replaced by a group —R^{L1}—, wherein said alkylene is optionally substituted with one or more groups R^{L2}, and optionally wherein one or more —CH₂— units in said alkylene are each replaced by a group independently selected from —O—, —NH—, —N(C₁₋₅ alkyl)-, —CO—, —S—, —SO— and —SO₂—.

R^{L1} is selected from —CO—N(R^{N})—, —N(R^{N})—CO—, —CO—O—, —O—CO—, —SO₂—N(R^{N})—, —N(R^{N})—SO₂—, —CO—O—N(R^{N})—, —N(R^{N})—CO—O—, —CS—N(R^{N})—, —N(R^{N})—CS—, —N(R^{N})—CO—N(R^{N})—, —C(═N(R^{N}))—N(R^{N})—, —N(R^{N})—C(═N(R^{N}))—, —C(—CF₃)—N(R^{N})—, —N(R^{N})—C(—CF₃)—, —O—P(═O)(C₁₋₅ alkyl)-N(R^{N})—, —N(R^{N})—P(═O)(C₁₋₅ alkyl)-O—, —CH═CH—, —CF═CH—, —CH═CF—, imidazoldiyl, thiazoldiyl, triazoldiyl, oxadiazoldiyl, tetrazoldiyl, diketopiperazindiyl, -oxetandiyl-N(R^{N})—, and —N(R^{N})-oxetandiyl-.

Each R^{N} is independently hydrogen or C₁₋₅ alkyl, wherein said alkyl is optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Each R^{L2} is independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{C1}-R^{C1}, —(C₀₋₃ alkylene)-cycloalkyl, and —(C₀₋₃ alkylene)-heterocycloalkyl.

Each L^{C1} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{C1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

The present invention also relates to a pharmaceutical composition comprising a compound of formula (I) or a pharmaceutically acceptable salt thereof, and a pharmaceutically acceptable excipient. Accordingly, the invention relates to a compound of formula (I) or a pharmaceutically acceptable salt thereof, or a pharmaceutical composition comprising any of the aforementioned entities and a pharmaceutically acceptable excipient, for use in therapy (or for use as a medicament).

The invention further relates to a compound of formula (I) or a pharmaceutically acceptable salt thereof, or a pharmaceutical composition comprising any of the aforementioned entities and a pharmaceutically acceptable excipient, for use in the treatment or prevention of an SLC16A3-associated disease/disorder (or an SLC16A3-mediated disease/disorder). Thus, the invention in particular provides a pharmaceutical composition comprising, as an active ingredient, a compound of formula (I) or a pharmaceutically acceptable salt thereof, together with a pharmaceutically acceptable excipient, for use in the treatment or prevention of an SLC16A3-associated disease/disorder.

Moreover, the present invention relates to the use of a compound of formula (I) or a pharmaceutically acceptable salt thereof in the preparation of a medicament for the treatment or prevention of an SLC16A3-associated disease/disorder.

The invention likewise relates to a method of treating or preventing an SLC16A3-associated disease/disorder, the method comprising administering a compound of formula (I) or a pharmaceutically acceptable salt thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt thereof (or of the pharmaceutical composition) is to be administered in accordance with this method.

As explained above, the disease or disorder to be treated or prevented with a compound of formula (I) or a pharmaceutically acceptable salt thereof (or a corresponding pharmaceutical composition) in accordance with the present invention encompasses any SLC16A3-associated disease/disorder, including also any of the diseases/disorders mentioned herein above in the introduction (background section) of the present specification. It is preferred that the disease/disorder to be treated or prevented in accordance with the invention is selected from cancer, an inflammatory disorder (e.g., rheumatoid arthritis or inflammatory bowel disease), a cardiovascular disorder (e.g., heart failure or cardiac hypertrophy), a fibrotic disorder (e.g., pulmonary fibrosis (including idiopathic pulmonary fibrosis), liver fibrosis, cystic fibrosis, renal fibrosis, peritoneal fibrosis, pancreatic fibrosis, intestinal fibrosis, cardiac fibrosis, or skin fibrosis), and Alzheimer's disease. The SLC16A3-associated disease/disorder to be treated or prevented may also be, e.g., muscular dystrophy, systemic sclerosis, metabolic syndrome, diabetes, dyslipidemia, fatty liver disease, non-alcoholic steatohepatitis, obesity, or insulin resistance. The invention particularly relates to the treatment or prevention of cancer.

The cancer to be treated or prevented in accordance with the present invention is preferably selected from lung cancer (e.g., non-small cell lung cancer or small cell lung cancer; particularly non-small cell lung cancer), cervical cancer (e.g., cervical squamous cell carcinoma or endocervical adenocarcinoma), colorectal cancer, colon cancer (e.g., colon adenocarcinoma), rectal cancer (e.g., rectum adenocarcinoma), glioblastoma (e.g., glioblastoma multiforme), gastric cancer (e.g., stomach adenocarcinoma), ovarian cancer (e.g., ovarian serous cystadenocarcinoma), head and neck squamous cell carcinoma, oral squamous cell carcinoma, breast cancer (e.g., invasive breast cancer, triple negative breast cancer, or HER2-positive breast cancer), prostate cancer (e.g., prostate adenocarcinoma), bladder cancer (e.g., bladder urothelial carcinoma), liver cancer (e.g., hepatocellular carcinoma), renal cancer (e.g., kidney renal clear cell carcinoma or kidney renal papillary cell carcinoma), thyroid cancer, pancreatic cancer (e.g., pancreatic neuroendocrine cancer, pancreatic adenocarcinoma, or pancreatic ductal adenocarcinoma), bone cancer (e.g., giant cell bone cancer), leukemia (e.g., acute myeloid leukemia (AML), chronic myeloid leukemia (CML), acute lymphocytic leukemia (ALL), or chronic lymphocytic leukemia (CLL); particularly AML), lymphoma (e.g., diffuse large B-cell lymphoma or non-Hodgkin lymphoma), melanoma (e.g., skin cutaneous melanoma), endometrial cancer (e.g., uterine corpus endometrial carcinoma), uterine sarcoma (e.g., uterine carcinosarcoma), and multiple myeloma. The cancer many also be, e.g., adult T-cell leukemia, blastoma, bone cancer, brain cancer, carcinoma, myeloid sarcoma, esophageal cancer, gastrointestinal cancer, glioma, gallbladder cancer, Hodgkin's lymphoma, intestinal cancer, laryngeal cancer, leukemia, lymphoma, mesothelioma, ocular cancer, optic nerve tumor, pituitary tumor, primary central nervous lymphoma, pharyngeal cancer, sarcoma, skin cancer, spinal tumor, small intestine cancer, T-cell lymphoma, testicular cancer, throat cancer, urogenital cancer, urothelial carcinoma, uterine cancer, vaginal cancer, or Wilms' tumor. More preferably, the cancer is selected from lung cancer, colorectal cancer, breast cancer, prostate cancer, renal cancer, and pancreatic cancer. The cancer (including any one of the specific cancers described above) may also be an angiogenesis inhibitor-resistant cancer. The cancer may also be a glycolysis-dependent cancer or a cancer which does not depend on glycolysis.

The compounds provided herein can also be used for re-sensitization of cancers/tumors to anti-angiogenic therapy. Accordingly, the invention also relates to a compound of formula (I) or a pharmaceutically acceptable salt thereof (or a corresponding pharmaceutical composition) for use in re-sensitizing a cancer (particularly a tumorous cancer) to anti-angiogenic therapy. The invention likewise relates to a compound of formula (I) or a pharmaceutically acceptable salt thereof (or a corresponding pharmaceutical composition) for use in treating or preventing cancer (including any of the specific cancers described herein above), wherein said compound (or said pharmaceutical composition) is administered in combination with an angiogenesis inhibitor.

The present invention furthermore relates to the use of a compound of formula (I) or a pharmaceutically acceptable salt thereof as an inhibitor of SLC16A3 (or MCT4) in research, particularly as a research tool compound for inhibiting SLC16A3. Accordingly, the invention refers to the in vitro use of a compound of formula (I) or a pharmaceutically acceptable salt thereof as an SLC16A3 inhibitor (or an MCT4 inhibitor) and, in particular, to the in vitro use of a compound of formula (I) or a pharmaceutically acceptable salt thereof as a research tool compound acting as an SLC16A3 inhibitor (or an MCT4 inhibitor). The invention likewise relates to a method, particularly an in vitro method, of inhibiting SLC16A3, the method comprising the application of a compound of formula (I) or a pharmaceutically acceptable salt thereof. The invention further relates to a method of inhibiting SLC16A3, the method comprising applying a compound of formula (I) or a pharmaceutically acceptable salt thereof to a test sample (e.g., a biological sample) or a test animal (i.e., a non-human test animal). The invention also refers to a method, particularly an in vitro method, of inhibiting SLC16A3 in a sample (e.g., a biological sample), the method comprising applying a compound of formula (I) or a pharmaceutically acceptable salt thereof to said sample. The present invention further provides a method of inhibiting SLC16A3, the method comprising contacting a test sample (e.g., a biological sample) or a test animal (i.e., a non-human test animal) with a compound of formula (I) or a pharmaceutically acceptable salt thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compounds according to the present invention, i.e. the compounds of formula (I) and the pharmaceutically acceptable salts thereof, will be described in more detail in the following:

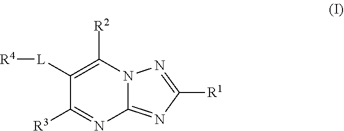

In formula (I), the group R¹ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two, three or four) groups R^{Cyc}, and further wherein one or more (e.g., one or two) —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

Preferably, R¹ is selected from hydrogen, C₁₋₅ alkyl, C₂₋₅ alkenyl, C₂₋₅ alkynyl, —(C₀₋₃ alkylene)-O(C₁₋₅ alkyl), —(C₀₋₃ alkylene)-S(C₁₋₅ alkyl), —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, R¹ is selected from hydrogen, C₁₋₅ alkyl, —O(C₁₋₅ alkyl), —S(C₁₋₅ alkyl), halogen, C₁₋₅ haloalkyl, —CN, carbocyclyl, and heterocyclyl, wherein said carbocyclyl and said heterocyclyl are each optionally substituted with one or more R^{Cyc}. Even more preferably, R¹ is selected from hydrogen, C₁₋₅ alkyl, —O(C₁₋₅ alkyl), —S(C₁₋₅ alkyl), halogen, C₁₋₅ haloalkyl, and —CN. Yet even more preferably, R¹ is selected from hydrogen, C₁₋₅ alkyl (e.g., methyl or ethyl), —O(C₁₋₅ alkyl) (e.g., —OCH₃ or —OCH₂CH₃), —S(C₁₋₅ alkyl) (e.g., —SCH₃ or —SCH₂CH₃) and C₁₋₅ haloalkyl (e.g., —CF₃). Particularly preferred examples of R¹ include —CH₃, —SCH₃ or —CF₃.

R² and R³ are each independently selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two, three or four) groups R^{Cyc}, and further wherein one or more (e.g., one or two) —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

Preferably, R² and R³ are each independently selected from hydrogen, C₁₋₅ alkyl, C₂₋₅ alkenyl, C₂₋₅ alkynyl, —(C₀₋₃ alkylene)-O(C₁₋₅ alkyl), —(C₀₋₃ alkylene)-S(C₁₋₅ alkyl), —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, R² and R³ are each independently selected from hydrogen, C₁₋₅ alkyl, —O(C₁₋₅ alkyl), —S(C₁₋₅ alkyl), halogen, C₁₋₅ haloalkyl, —CN, carbocyclyl, and heterocyclyl, wherein said carbocyclyl and said heterocyclyl are each optionally substituted with one or more R^{Cyc}. Even more preferably, R² and R³ are each independently selected from hydrogen, C₁₋₅ alkyl (e.g., methyl or ethyl), —O(C₁₋₅ alkyl) (e.g., —OCH₃ or —OCH₂CH₃), —S(C₁₋₅ alkyl) (e.g., —SCH₃ or —SCH₂CH₃), halogen, C₁₋₅ haloalkyl (e.g., —CF₃), and —CN. It is particularly preferred that R² and R³ are each independently C₁₋₅ alkyl; yet even more preferably, R² and R³ are each methyl.

R⁴ is aryl or heteroaryl, wherein said aryl or said heteroaryl is optionally substituted with one or more (e.g., one, two, three or four) groups R⁴¹, and further wherein said heteroaryl is not 1,2,4-triazolyl.

As explained above, R⁴ is aryl or heteroaryl (each of which is optionally substituted with one or more R⁴¹, and wherein said heteroaryl is different from 1,2,4-triazolyl). Said aryl is preferably phenyl or naphthyl (e.g., naphth-1-yl or naphth-2-yl), more preferably phenyl. Said heteroaryl is preferably a monocyclic heteroaryl or a bicyclic heteroaryl, more preferably a monocyclic heteroaryl (e.g., pyridinyl). Accordingly, it is preferred that R⁴ is selected from phenyl, monocyclic heteroaryl (e.g., pyridinyl), naphthyl and bicyclic heteroaryl (e.g., quinolinyl, such as quinolin-3-yl), wherein said phenyl, said monocyclic heteroaryl, said naphthyl and said bicyclic heteroaryl are each optionally substituted with one or more (e.g., one, two, three or four) groups R⁴¹, and wherein said monocyclic heteroaryl is not 1,2,4-triazolyl. If R⁴ is a monocyclic heteroaryl (which is optionally substituted with one or more R⁴¹), it is preferred that said monocyclic heteroaryl is a 5-membered or a 6-membered heteroaryl, particularly a 6-membered heteroaryl. If R⁴ is a fused bicyclic heteroaryl (which is optionally substituted with one or more R⁴¹), it is preferred that at least the ring (of said fused bicyclic heteroaryl) that is directly attached to group L is aromatic (such as, e.g., 5,6,7,8-tetrahydroquinolin-2-yl, 5,6,7,8-tetrahydroquinolin-3-yl, 5,6,7,8-tetrahydroquinolin-4-yl, 1,3-benzodioxol-4-yl or 1,3-benzodioxol-5-yl), and it is particularly preferred that both rings (of said fused bicyclic heteroaryl) are aromatic (such as, e.g., quinolin-3-yl or quinolin-7-yl). More preferably, R⁴ is phenyl or monocyclic heteroaryl, wherein said phenyl or said monocyclic heteroaryl is optionally substituted with one or more R⁴¹, and wherein said monocyclic heteroaryl is not 1,2,4-triazolyl. Even more preferably, R⁴ is phenyl or a 6-membered monocyclic heteroaryl, wherein said phenyl or said 6-membered monocyclic heteroaryl is optionally substituted with one or more R⁴¹. Yet even more preferably, R⁴ is phenyl or pyridinyl (e.g., pyridin-2-yl, pyridin-3-yl or pyridin-4-yl), wherein said phenyl or said pyridinyl is optionally substituted with one or more R⁴¹.

Particularly preferred examples of R⁴ include: (i) phenyl which is optionally substituted with one or more groups R⁴¹, wherein at least one group R⁴¹ is present and is attached in 3-position or 4-position of the phenyl ring; (ii) phenyl which is substituted with one group R⁴¹ attached in 3-position of the phenyl ring; (iii) phenyl which is substituted with one group R⁴¹ attached in 4-position of the phenyl ring; (iv) phenyl which is substituted with two groups R⁴¹ attached in 3-position and 4-position of the phenyl ring; (v) pyridinyl which is optionally substituted with one or more groups R⁴¹, wherein at least one group R⁴¹ is present and is attached in meta or para position of the pyridinyl ring (with respect to the attachment point of group L on the pyridinyl ring); (vi) pyridin-2-yl which is substituted with one group R⁴¹ attached in meta position (i.e., in 4-position or 6-position) of the pyridin-2-yl ring; (vii) pyridin-2-yl which is substituted with one group R⁴¹ attached in para position (i.e., in 5-position) of the pyridin-2-yl ring; (viii) pyridin-2-yl which is substituted with two groups R⁴¹ attached in meta position (i.e., in 4-position or 6-position) and in para position (i.e., in 5-position) of the pyridin-2-yl ring; (ix) pyridin-3-yl which is substituted with one group R⁴¹ attached in 5-position of the pyridin-3-yl ring; (x) pyridin-3-yl which is substituted with one group R⁴¹ attached in 6-position of the pyridin-3-yl ring; (xi) pyridin-3-yl which is substituted with two groups R⁴¹ attached in 5-position and in 6-position of the pyridin-3-yl ring; or (xii) pyridin-4-yl which is substituted with one group R⁴¹ attached in meta position (i.e., in 2-position or 6-position) of the pyridin-4-yl ring.

Each R⁴¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two, three or four) groups R^{Cyc}, and further wherein one or more (e.g., one or two) —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—.

Preferably, each R⁴¹ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —(C₀₋₃ alkylene)-CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—. More preferably, each R⁴¹ is independently selected from C₁₋₅ alkyl, C₂₋₅ alkenyl, C₂₋₅ alkynyl, —O(C₁₋₅ alkyl), —S(C₁₋₅ alkyl), —NH(C₁₋₅ alkyl), —N(C₁₋₅ alkyl)(C₁₋₅ alkyl), halogen, C₁₋₅ haloalkyl, —O—(C₁₋₅ haloalkyl), —(C₀₋₃ alkylene)-CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—. Even more preferably, each R⁴¹ is independently selected from C₁₋₅ alkyl, —O(C₁₋₅ alkyl), —S(C₁₋₅ alkyl), halogen, C₁₋₅ haloalkyl, —O—(C₁₋₅ haloalkyl), —CN, —(C₀₋₃ alkylene)-carbocyclyl, and —(C₀₋₃ alkylene)-heterocyclyl, wherein the carbocyclyl in said —(C₀₋₃ alkylene)-carbocyclyl and the heterocyclyl in said —(C₀₋₃ alkylene)-heterocyclyl are each optionally substituted with one or more groups R^{Cyc}. Yet even more preferably, each R⁴¹ is independently selected from C₁₋₅ alkyl (e.g., methyl or ethyl), —O(C₁₋₅ alkyl), —S(C₁₋₅ alkyl), halogen, C₁₋₅ haloalkyl, —O—(C₁₋₅ haloalkyl), and —CN.

L is C₂₋₈ alkylene, wherein one —CH₂— unit in said alkylene is replaced by a group —R^{L1}—, wherein said alkylene is optionally substituted with one or more (e.g., one, two or three) groups R^{L2}, and optionally wherein one or more (e.g., one, two or three) —CH₂— units in said alkylene are each replaced by a group independently selected from —O—, —NH—, —N(C₁₋₅ alkyl)-, —CO—, —S—, —SO— and —SO₂—.

Preferably, L is C₂₋₈ alkylene, wherein one —CH₂— unit in said alkylene is replaced by a group —R^{L1}—, and wherein said alkylene is optionally substituted with one or more groups R^{L2}. The group —R^{L1}- is preferably present in the indicated orientation; thus, for example, if R^{L1} is —CO—N(R^{N})—, then L may be, e.g., a group —(CH₂)ₓ−CO—N(R^{N})—(CH₂)_{y}—, wherein the variables x and y are each independently an integer of 0 to 7, provided that the sum of x+y is an integer of 1 to 7, wherein said group is attached via its —(CH₂)ₓ— moiety to the 1,2,4-triazolo[1,5-a]pyrimidine ring comprised in the compound of formula (I), and wherein said group is attached via its —(CH₂)_{y}— moiety to R⁴; conversely, if R^{L1} is —N(R^{N})—CO—, then L may be, e.g., a group —(CH₂)ₓ—N(R^{N})—CO—(CH₂)_{y}—, wherein the variables x and y are each independently an integer of 0 to 7, provided that the sum of x+y is an integer of 1 to 7, wherein said group is attached via its —(CH₂)ₓ— moiety to the 1,2,4-triazolo[1,5-a]pyrimidine ring comprised in the compound of formula (I), and wherein said group is attached via its —(CH₂)_{y}— moiety to R⁴. The aforementioned variable x is preferably 1 to 4, more preferably 2 or 3, even more preferably 2; the aforementioned variable y is preferably 0 to 3, more preferably 0, 1 or 2, even more preferably 0. Moreover, it is preferred that said C₂₋₈ alkylene is a C₂₋₆ alkylene, particularly a C₂₋₅ alkylene (wherein one —CH₂— unit in said alkylene is replaced by a group —R^{L1}—, as explained above).

More preferably, L is a group —(C₁₋₄ alkylene)-R^{L1}—, wherein said alkylene is optionally substituted with one or more R^{L2}, and wherein said group is attached via its —R^{L1}— moiety to R⁴. Said C₁₋₄ alkylene is preferably selected from —CH₂—, —CH₂CH₂—, —CH₂CH₂CH₂— and —CH₂CH₂CH₂CH₂—, more preferably from —CH₂CH₂— and —CH₂CH₂CH₂—, and is even more preferably —CH₂CH₂—. It is furthermore preferred that said alkylene is unsubstituted, i.e., is not substituted with any group R^{L2}.

R^{L1} is selected from —CO—N(R^{N})—, —N(R^{N})—CO—, —CO—O—, —O—CO—, —SO₂—N(R^{N})—, —N(R^{N})—SO₂—, —CO—O—N(R^{N})—, —N(R^{N})—CO—O—, —CS—N(R^{N})—, —N(R^{N})—CS—, —N(R^{N})—CO—N(R^{N})—, —C(═N(R^{N}))—N(R^{N})—, —N(R^{N})—C(═N(R^{N}))—, —C(—CF₃)—N(R^{N})—, —N(R^{N})—C(—CF₃)—, —O—P(═O)(C₁₋₅ alkyl)-N(R^{N})—, —N(R^{N})—P(═O)(C₁₋₅ alkyl)-O—, —CH═CH—, —CF═CH—, —CH═CF—, imidazoldiyl, thiazoldiyl, triazoldiyl, oxadiazoldiyl, tetrazoldiyl, diketopiperazindiyl, -oxetandiyl-N(R^{N})—, and —N(R^{N})-oxetandiyl-.

Preferably, R^{L1} is selected from —CO—N(R^{N})—, —N(R^{N})—CO—, —CO—O—, —O—CO—, —SO₂—N(R^{N})—, —N(R^{N})—SO₂—, —CO—O—N(R^{N})—, —N(R^{N})—CO—O—, —CS—N(R^{N})—, —N(R^{N})—CS—, —N(R^{N})—CO—N(R^{N})—, —C(═N(R^{N}))—N(R^{N})—, —N(R^{N})—C(═N(R^{N}))—, imidazoldiyl (e.g., imidazol-2,5-diyl, imidazol-2,4-diyl, imidazol-1,2-diyl, imidazol-1,5-diyl, imidazol-1,4-diyl, or imidazol-4,5-diyl), thiazoldiyl (e.g., thiazol-2,4-diyl, thiazol-2,5-diyl, or thiazol-4,5-diyl), triazoldiyl (particularly 1,2,3-triazoldiyl or 1,2,4-triazoldiyl; e.g., 1H-1,2,3-triazol-1,4-diyl, 1H-1,2,3-triazol-1,5-diyl, 1H-1,2,3-triazol-4,5-diyl, 2H-1,2,3-triazol-2,4-diyl, 1H-1,2,4-triazol-1,3-diyl, 1H-1,2,4-triazol-3,5-diyl, 1H-1,2,4-triazol-1,5-diyl, 4H-1,2,4-triazol-3,4-diyl, or 4H-1,2,4-triazol-4,5-diyl), oxadiazoldiyl (particularly 1,2,4-oxadiazoldiyl, 1,2,5-oxadiazoldiyl, or 1,3,4-oxadiazoldiyl; e.g., 1,2,4-oxadiazol-3,5-diyl, 1,2,5-oxadiazol-3,4-diyl, or 1,3,4-oxadiazol-2,5-diyl), tetrazoldiyl (e.g., 1H-tetrazol-1,5-diyl or 2H-tetrazol-2,5-diyl), diketopiperazindiyl (e.g., 2,5-diketopiperazin-1,3-diyl), -oxetandiyl-N(R^{N})— (e.g., -oxetan-3,3-diyl-N(R^{N})—), and —N(R^{N})-oxetandiyl- (e.g., —N(R^{N})-oxetan-3,3-diyl-). More preferably, R^{L1} is selected from —CO—N(R^{N})—, —N(R^{N})—CO—, —CO—O—, —O—CO—, —SO₂—N(R^{N})—, —N(R^{N})—SO₂—, —CO—O—N(R^{N})—, —N(R^{N})—CO—O—, and —N(R^{N})—CO—N(R^{N})—. Even more preferably, R^{L1} is —CO—N(R^{N})— or —N(R^{N})—CO—, particularly —CO—N(R^{N})—. Accordingly, it is particularly preferred that R^{L1} is —CO—N(R^{N})—, wherein the —N(R^{N})— moiety of said —CO—N(R^{N})— is more proximal (i.e., closer) to R⁴ and the —CO— moiety of said —CO—N(R^{N})— is more distant (i.e., less close) to R⁴.

In accordance with the above preferred definitions of L and R^{L1}, it is particularly preferred that L is a group —CH₂CH₂— CO—N(R^{N})— which is attached via its —CO—N(R^{N})— moiety to R⁴ (and via its —CH₂CH₂— moiety to the 1,2,4-triazolo[1,5-a]pyrimidine ring comprised in the compound of formula (I)). Accordingly, it is particularly preferred that the compound of formula (I) has the following structure:

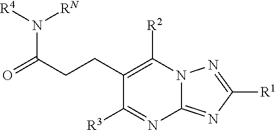

Each R^{N} is independently hydrogen or C₁₋₅ alkyl, wherein said alkyl is optionally substituted with one or more (e.g., one, two or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Preferably, each R^{N} is independently hydrogen or C₁₋₅ alkyl (e.g., methyl or ethyl). For example, R^{N} may be hydrogen.

Each R^{L2} is independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

Preferably, each R^{L2} is independently selected from halogen, C₁₋₅ haloalkyl, —O—(C₁₋₅ haloalkyl), —CN, —O(C₁₋₅ alkyl), —S(C₁₋₅ alkyl), —NH(C₁₋₅ alkyl), and —N(C₁₋₅ alkyl)(C₁₋₅ alkyl). More preferably, each R^{L2} is independently selected from halogen, C₁₋₅ haloalkyl, —O—(C₁₋₅ haloalkyl), —CN, —O(C₁₋₅ alkyl), and —S(C₁₋₅ alkyl).

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{C1}-R^{C1}, —(C₀₋₃ alkylene)-cycloalkyl, and —(C₀₋₃ alkylene)-heterocycloalkyl.

Each L^{C1} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more (e.g., one, two or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{C1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two, three or four) groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

It is particularly preferred that the compound of formula (I) is any one of the specific compounds described herein below in the examples section, either in non-salt form or as a pharmaceutically acceptable salt of the respective compound.

Accordingly, it is particularly preferred that the compound of formula (I) is any one of the following compounds or a pharmaceutically acceptable salt thereof:

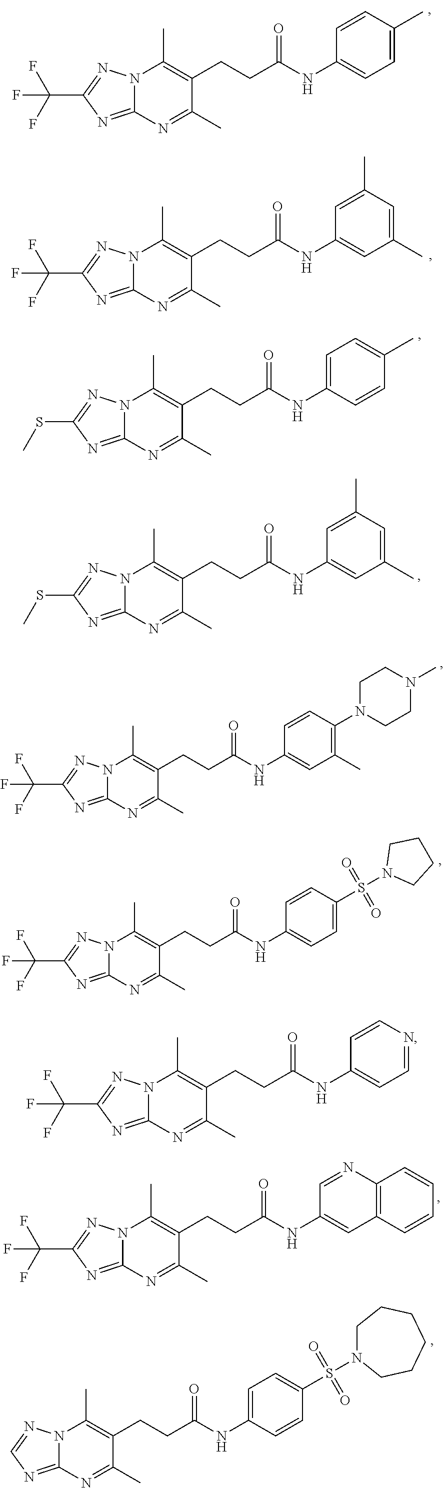

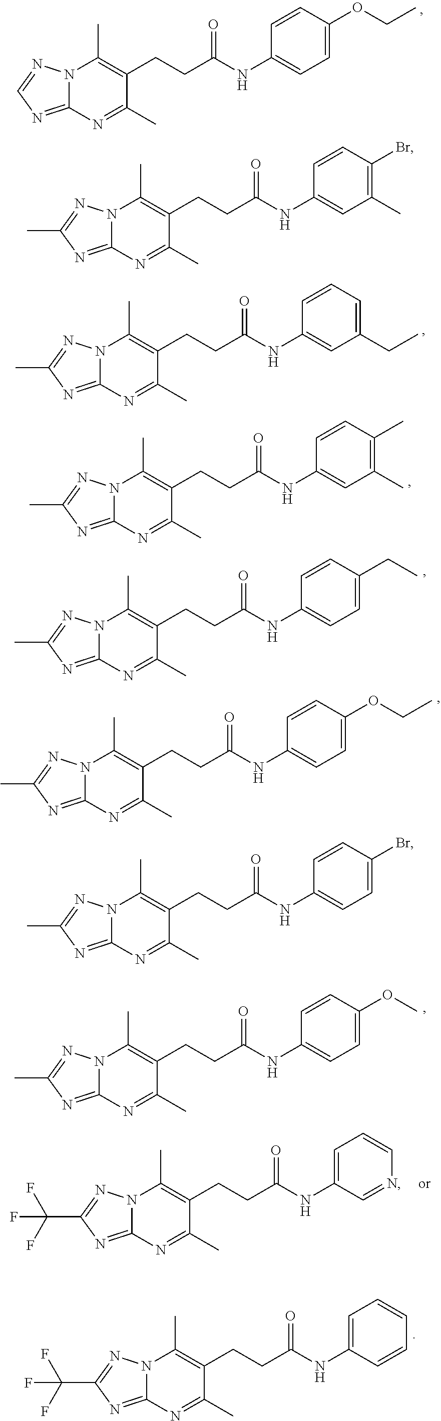

For a person skilled in the field of synthetic chemistry, various ways for the preparation of the compounds of formula (I) and their pharmaceutically acceptable salts will be readily apparent. For example, the compounds of formula (I) can be prepared in accordance with, or in analogy to, the synthetic routes described in the following general schemes (abbreviations used—“AcOH”: acetic acid; “LAH”: lithium aluminum hydride; “THF”: tetrahydrofuran; “TCT”: 2,4,6-trichloro-1,3,5-triazine; “nBuLi”: n-butyllithium; “TEA” or “NEt₃”: triethylamine; “PPh₃”: triphenylphosphine; “DEAD”: diethyl azodicarboxylate; the numbering of the variable groups in these synthetic schemes may vary from the numbering used in formula (I)):

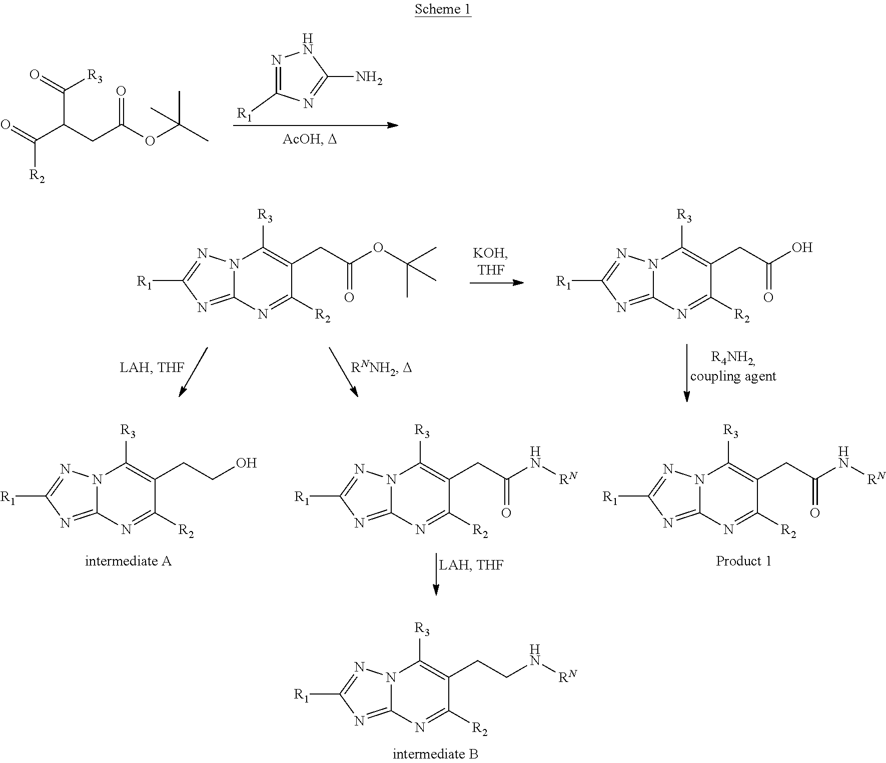

### 1—General synthesis of 2-([1,2,4]triazolo[1,5-a]pyrimidin-6-yl)acetamides (Product 1)

Suitably-substituted tert-butyl 3-formyl-4-oxobutanoate is reacted with 3-substituted 1H-1,2,4-triazol-5-amine to give [2,5,7]-trisubstituted tert-butyl 2-([1,2,4]triazolo[1,5-a]pyrimidin-6-yl)acetate, which is converted in the corresponding acid via basic hydrolysis. Amide coupling with commercially-available substitute primary amine R₄NH₂ and a suitable coupling agent affords 2-([1,2,4]triazolo[1,5-a]pyrimidin-6-yl)acetamides (Product 1).

### 2—General synthesis of 2-([1,2,4]triazolo[1,5-a]pyrimidin-6-yl)ethan-1-ols (Intermediate A)

Suitably-substituted tert-butyl 3-formyl-4-oxobutanoate is reacted with 3-substituted 1H-1,2,4-triazol-5-amine to give [2,5,7]-trisubstituted tert-butyl 2-([1,2,4]triazolo[1,5-a]pyrimidin-6-yl)acetate which is reduced with lithium aluminum hydride (LAH) to give the corresponding primary alcohol 2-([1,2,4]triazolo[1,5-a]pyrimidin-6-yl)ethan-1-ols (Intermediate A).

### 3—General synthesis of 2-([1,2,4]triazolo[1,5-a]pyrimidin-6-yl)ethan-1-amines (Intermediate B)

Suitably-substituted tert-butyl 3-formyl-4-oxobutanoate is reacted with 3-substituted 1H-1,2,4-triazol-5-amine to give [2,5,7]-trisubstituted tert-butyl 2-([1,2,4]triazolo[1,5-a]pyrimidin-6-yl)acetate which is reacted with a suitably-substituted primary amine (R^{N}NH₂) to give the corresponding secondary amide, which is then reduced with lithium aluminum hydride (LAH) to give the corresponding primary amine 2-([1,2,4]triazolo[1,5-a]pyrimidin-6-yl)ethan-1-amines (Intermediate B).

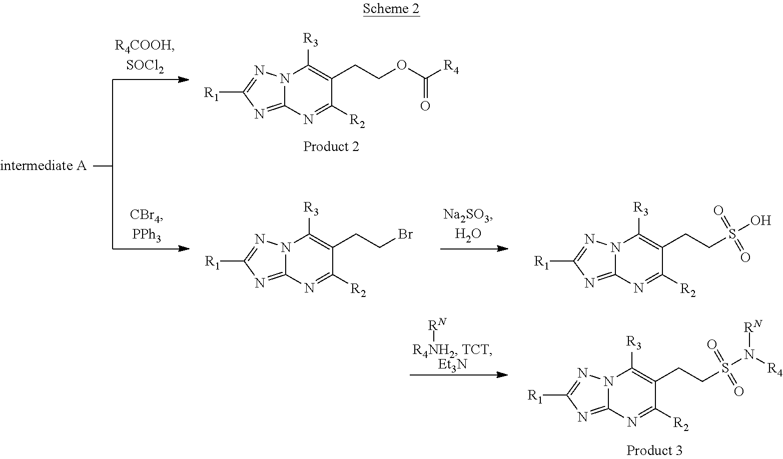

### 1—General synthesis of 2-([1,2,4]triazolo[1,5-a]pyrimidin-6-yl)ethan-1-ol esters (Product 2)

Intermediate A is reacted with thionyl chloride (SOCl₂) and a suitably-substituted carboxylic acid (R₄COOH) to yield the corresponding 2-([1,2,4]triazolo[1,5-a]pyrimidin-6-yl)ethan-1-ol esters (Product 2).

### 2—General synthesis of 2-([1,2,4]triazolo[1,5-a]pyrimidin-6-yl)ethane-1-sulfonamides (Product 3)

Intermediate A undergoes Appel reaction with CBr₄ to give the corresponding primary alkyl bromide, which is treated with Na₂SO₃ to afford the corresponding sulfonic acid. Reaction with a suitably-substituted secondary amine R₄(R^{N})NH₂ yields 2-([1,2,4]triazolo[1,5-a]pyrimidin-6-yl)ethane-1-sulfonamides (Product 3).

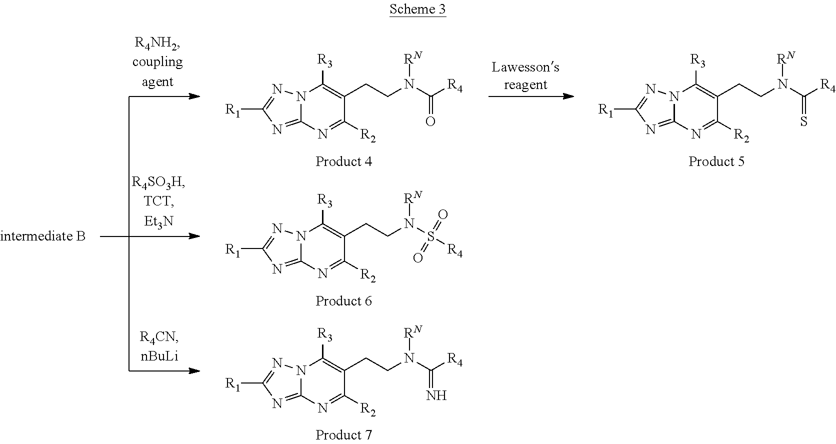

### 1—General synthesis of 2-([1,2,4]triazolo[1,5-a]pyrimidin-6-yl)ethan-1-amine carboxamides (Product 4)

Intermediate B is reacted with a suitably-substituted carboxylic acid (R₄COOH) to give the corresponding 2-([1,2,4]triazolo[1,5-a]pyrimidin-6-yl)ethan-1-amine carboxamides (Product 4).

### 2—General synthesis of 2-([1,2,4]triazolo[1,5-a]pyrimidin-6-yl)ethan-1-amine thioamides (Product 5)

Product 4 is converted in the corresponding thioamide by treatment with Lawesson's reagent to give 2-([1,2,4]triazolo[1,5-a]pyrimidin-6-yl)ethan-1-amine thioamides (Product 5).

### 3—General synthesis of 2-([1,2,4]triazolo[1,5-a]pyrimidin-6-yl)ethan-1-amine sulfonamides (Product 6)

Intermediate B is reacted with a suitably-substituted sulfonic acid (R₄SO₃H) to give the 2-([1,2,4]triazolo[1,5-a]pyrimidin-6-yl)ethan-1-amine sulfonamides (Product 6).

### 4—General synthesis of 2-([1,2,4]triazolo[1,5-a]pyrimidin-6-yl)ethan-1-amine imidamides (Product 7)

Intermediate B is reacted with a suitably-substituted nitrile (R₄CN) in strong basic conditions to give the corresponding 2-([1,2,4]triazolo[1,5-a]pyrimidin-6-yl)ethan-1-amine imidamides (Product 7).

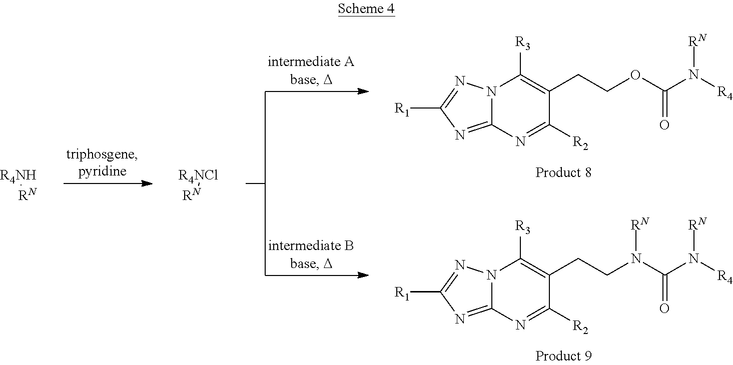

### 1) General synthesis of 2-([1,2,4]triazolo[1,5-a]pyrimidin-6-yl)ethan-1-ol carbamates (Product 8)

A suitably-substituted secondary amine R₄(R^{N})NH₂ is converted in the corresponding isocyanate by reaction with triphosgene and pyridine. The latter is reacted with Intermediate A to give the corresponding 2-([1,2,4]triazolo[1,5-a]pyrimidin-6-yl)ethan-1-ol carbamates (Product 8).

### 2) General synthesis of 1-(2-([1,2,4]triazolo[1,5-a]pyrimidin-6-yl)ethyl)ureas (Product 9)

A suitably-substituted secondary amine R₄(R^{N})NH₂ is converted in the corresponding isocyanate by reaction with triphosgene and pyridine. The latter is reacted with Intermediate B to give the corresponding 1-(2-([1,2,4]triazolo[1,5-a]pyrimidin-6-yl)ethyl)ureas (Product 9).

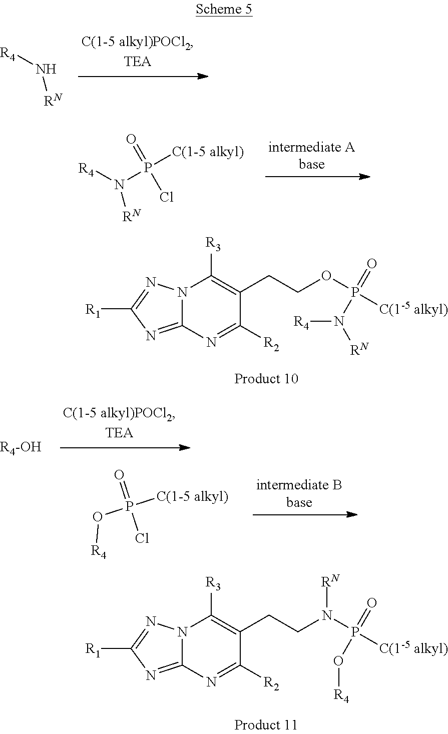

### 1) General synthesis of P—C₍₁₋₅₎alkyl-2-([1,2,4]triazolo[1,5-a]pyrimidin-6-yl)ethylphosphonamidates (Product 10)

A suitably-substituted secondary amine R₄(R^{N})NH₂ is reacted with (C₁₋₅)alkylphosphonic dichloride to afford the corresponding N,N-disubstituted-P—C₍₁₋₅₎alkyl-phosphonamidic chloride, which is converted in the corresponding P—C₍₁₋₅₎alkyl-2-([1,2,4]triazolo[1,5-a]pyrimidin-6-yl)ethylphosphonamidates (Product 10) by reaction with Intermediate A under basic conditions.

### 2) General synthesis of N-(2-([1,2,4]triazolo[1,5-a]pyrimidin-6-yl)ethyl)-P—C₍₁₋₅₎alkylphosphonamidic esters (Product 11)

A suitably-substituted primary alcohol R₄OH is reacted with (C₁₋₅)alkylphosphonic dichloride to afford the corresponding P—C₍₁₋₅₎alkyl phosphonochloridric acid ester, which is converted in the corresponding N-(2-([1,2,4]triazolo[1,5-a]pyrimidin-6-yl)ethyl)-P—C₍₁₋₅₎alkylphosphonamidic esters (Product 11) by reaction with Intermediate B under basic conditions.

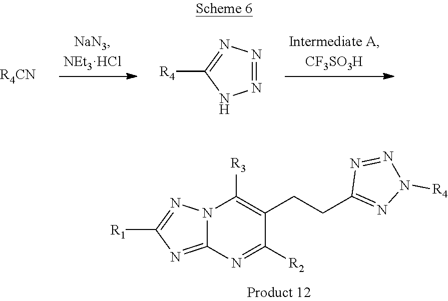

### General synthesis of 6-(2-(2H-tetrazol-5-yl)ethyl)-[1,2,4]triazolo[1,5-a]pyrimidines (Product 12)

Commercially-available nitrile R₄CN is reacted with NaN₃ and triethylamine hydrochloride to give the corresponding 5-substituted tetrazole derivative. The reaction with Intermediate A in presence of CF₃SO₃H affords the corresponding 6-(2-(2H-tetrazol-5-yl)ethyl)-[1,2,4]triazolo[1,5-a]pyrimidines (Product 12).

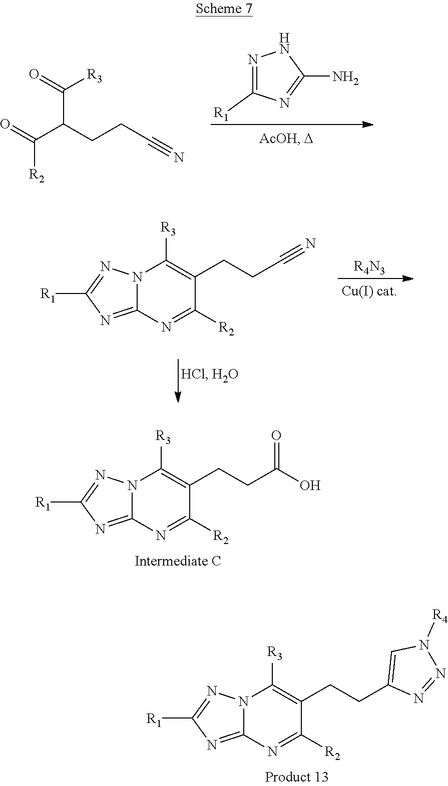

### 1—General synthesis of 6-(2-(1H-1,2,3-triazol-4-yl)ethyl)-[1,2,4]triazolo[1,5-a]pyrimidines (Product 13)

Suitably substituted 4-formyl-5-oxopentanenitrile is reacted with a 3-substituted 1H-1,2,4-triazol-5-amine under acidic conditions to give the corresponding 3-([1,2,4]triazolo[1,5-a]pyrimidin-6-yl)propanenitrile. Cu(I) catalyzed reaction with substituted azide R₄N₃ yields the corresponding 6-(2-(1H-1,2,3-triazol-4-yl)ethyl)-[1,2,4]triazolo[1,5-a]pyrimidines (Product 13).

### 2—General synthesis 3-([1,2,4]triazolo[1,5-a]pyrimidin-6-yl)propanoic acids (Intermediate C)

Suitably substituted 4-formyl-5-oxopentanenitrile is reacted with a 3-substituted 1H-1,2,4-triazol-5-amine under acidic conditions to give the corresponding 3-([1,2,4]triazolo[1,5-a]pyrimidin-6-yl)propanenitrile, which is hydrolyzed in acidic conditions to afford the corresponding 3-([1,2,4]triazolo[1,5-a]pyrimidin-6-yl)propanoic acids (Intermediate C).

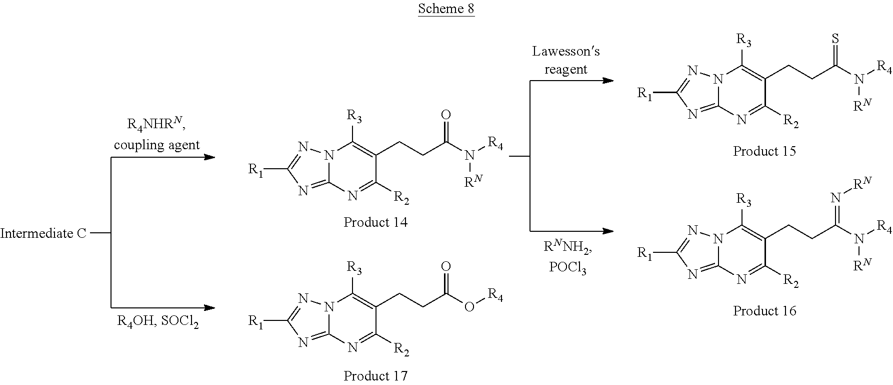

### 1—General synthesis of 3-([1,2,4]triazolo[1,5-a]pyrimidin-6-yl)propanoic carboxamides (Product 14)

Intermediate C is reacted with a suitably-substituted secondary amine R₄(R^{N})NH₂ in presence of a suitable amide coupling agent to afford 3-([1,2,4]triazolo[1,5-a]pyrimidin-6-yl)propanoic carboxamides (Product 14).

### 2—General synthesis of 3-([1,2,4]triazolo[1,5-a]pyrimidin-6-yl)propanethioamides (Product 15)

Product 14 is treated with Lawesson's reagent to give corresponding thioamides 3-([1,2,4]triazolo[1,5-a]pyrimidin-6-yl)propanethioamides (Product 15).

### 3—General synthesis of 3-([1,2,4]triazolo[1,5-a]pyrimidin-6-yl)propanimidamides (Product 16)

Product 14 is reacted with suitably-substituted primary amine R^{N}NH₂ and POCl₃ to give the corresponding 3-([1,2,4]triazolo[1,5-a]pyrimidin-6-yl)propanimidamides (Product 16).

### 4—General synthesis of 3-([1,2,4]triazolo[1,5-a]pyrimidin-6-yl)propanoic acid esters (Product 17)

Intermediate C is reacted with SOCl2 and a suitably-substituted alcohol R₄OH to give the corresponding 3-([1,2,4]triazolo[1,5-a]pyrimidin-6-yl)propanoic acid esters (Product 17).

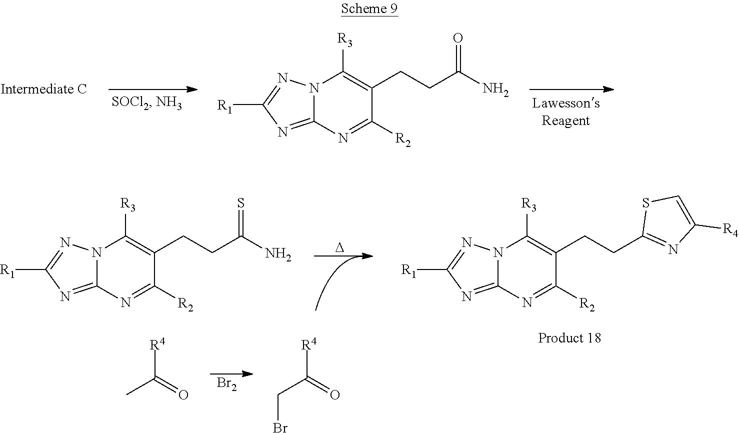

### General synthesis of 2-(2-([1,2,4]triazolo[1,5-a]pyrimidin-6-yl)ethyl)thiazoles (Product 18)

Intermediate C is reacted with NH₃ and SOCl₂. The resulting primary amide is converted into the corresponding thioamide by reaction with Lawesson's reagent and subsequently reacted with a suitably-substituted alpha-bromoketone, directly obtained from the bromination of substituted ketone R₄COCH₃, to give corresponding 2-(2-([1,2,4]triazolo[1,5-a]pyrimidin-6-yl)ethyl)thiazoles (Product 18).

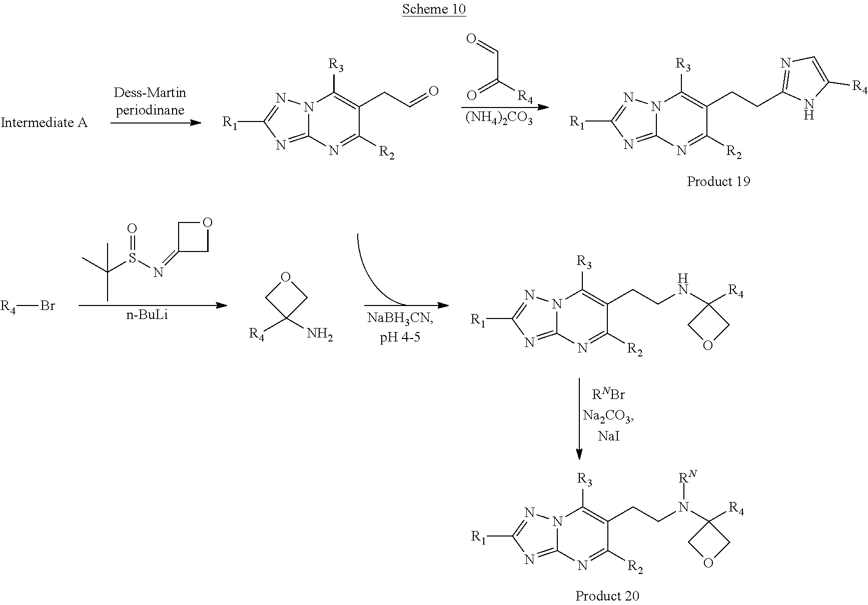

### 1—General synthesis of 6-(2-(1H-imidazol-2-yl)ethyl)-[1,2,4]triazolo[1,5-a]pyrimidines (Product 19)

Intermediate A is oxidized to the corresponding aldehyde with Dess-Martin periodinane and then treated with mono- or di-substituted glyoxals and ammonium carbonate to give the corresponding 6-(2-(1H-imidazol-2-yl)ethyl)-[1,2,4]triazolo[1,5-a]pyrimidines (Product 19).

### 2—General synthesis of N-(2-([1,2,4]triazolo[1,5-a]pyrimidin-6-yl)ethyl)oxetan-3-amines (Product 20)

Intermediate A is oxidized to the corresponding aldehyde with Dess-Martin periodinane. The product is then reacted with a suitably-substituted oxetan-3-amine, obtained from the corresponding alkyl bromide R₄Br by reaction with 2-methyl-N-(oxetan-3-ylidene)propane-2-sulfinamide and nBuLi, in a reductive amination reaction mediated by NaBH₃CN. The obtained secondary N-(2-([1,2,4]triazolo[1,5-a]pyrimidin-6-yl)ethyl)oxetan-3-amine is alkylated on the aminooxetane nitrogen to afford the corresponding tertiary N-(2-([1,2,4]triazolo[1,5-a]pyrimidin-6-yl)ethyl)oxetan-3-amines (Product 20).

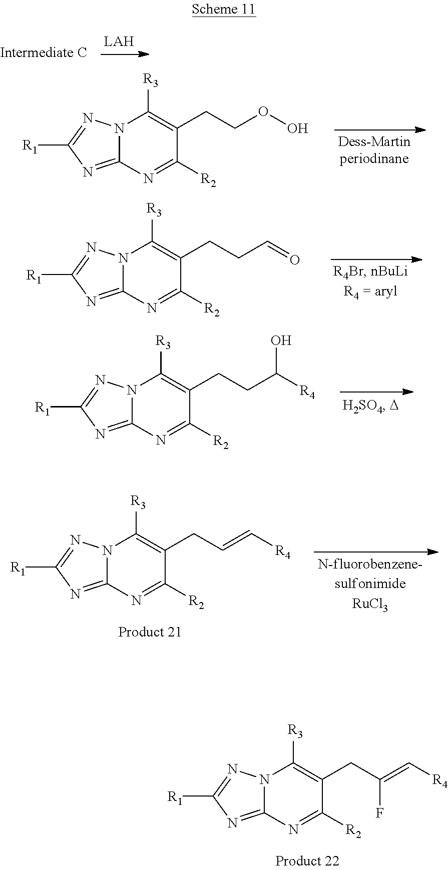

### 1—General synthesis of 6-arylallyl-[1,2,4]triazolo[1,5-a]pyrimidines (Product 21)

Intermediate C is reduced to the corresponding primary alcohol with LAH and oxidized to aldehyde with Dess-Martin periodinane. Reaction with the aryllithium salt formed by treating a suitably-substituted arylbromide R₄Br with nBuLi gives the corresponding secondary alcohol, which is dehydrated with H₂SO₄ to yield the corresponding arylally derivatives 6-arylallyl-[1,2,4]triazolo[1,5-a]pyrimidines (Product 21).

### 2—General synthesis of 6-(2-fluoro-3-aryllallyl)-[1,2,4]triazolo[1,5-a]pyrimidines (Product 22)

Product 21 is reacted with N-fluorobenzene-sulfonimide in presence of RuCl3₃ to give the corresponding 6-(2-fluoro-3-aryllallyl)-[1,2,4]triazolo[1,5-a]pyrimidines (Product 22).

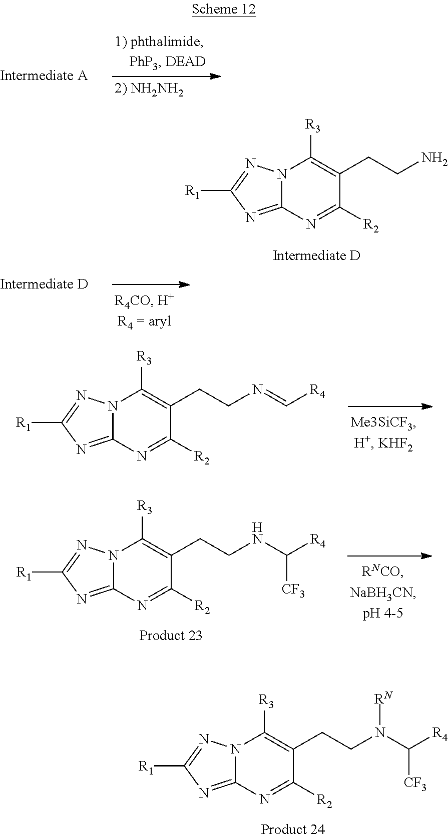

### 1—General synthesis of 2-([1,2,4]triazolo[1,5-a]pyrimidin-6-yl)ethan-1-amines (Intermediate D)

Gabriel amine synthesis converts Intermediate A in the corresponding terminal amine by reaction with phthalimide in presence of PPh₃ and DEAD followed by treatment with hydrazine, to give 2-([1,2,4]triazolo[1,5-a]pyrimidin-6-yl)ethan-1-amines (Intermediate D).

### 2—General synthesis of secondary N-(2-([1,2,4]triazolo[1,5-a]pyrimidin-6-yl)ethyl)-2,2,2-trifluoro-1-arylethan-1-amines (Product 23) and tertiary N-(2-([1,2,4]triazolo[1,5-a]pyrimidin-6-yl)ethyl)-2,2,2-trifluoro-1-arylethan-1-amines (Product 24)

Intermediate D is reacted with a suitably-substituted aldehyde R₄CO under acid catalysis to give the corresponding N-(2-([1,2,4]triazolo[1,5-a]pyrimidin-6-yl)ethyl)-1-arylmethanimine. Reaction with Me₃SiCF₃ in presence of acid and KHF₂ yields secondary N-(2-([1,2,4]triazolo[1,5-a]pyrimidin-6-yl)ethyl)-2,2,2-trifluoro-1-arylethan-1-amines (Product 23). N-alkylation of Product 23 affords tertiary N-(2-([1,2,4]triazolo[1,5-a]pyrimidin-6-yl)ethyl)-2,2,2-trifluoro-1-arylethan-1-amines (Product 24).

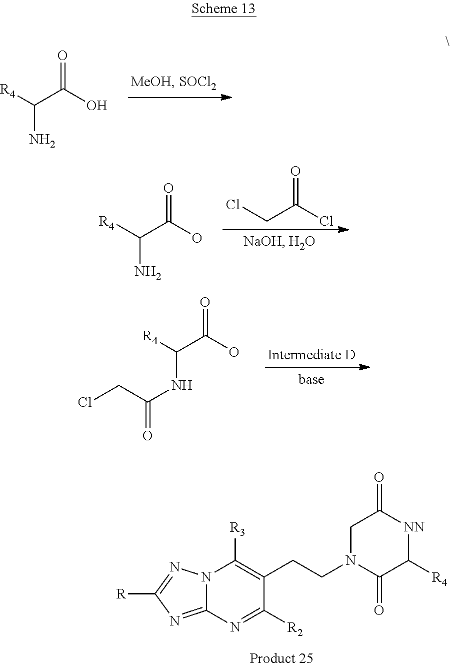

### General synthesis of 1-(2-([1,2,4]triazolo[1,5-a]pyrimidin-6-yl)ethyl)piperazine-2,5-diones (Product 25)

Suitably-substituted 2-aminoacetic acid is converted into the corresponding methyl ester by treatment with SOCl₂ in methanol and then in the corresponding methyl 2-(2-chloroacetamido)-acetate by reaction with 2-chloroacetyl chloride. The latter is reacted with Intermediate D in basic conditions to give the corresponding 1-(2-([1,2,4]triazolo[1,5-a]pyrimidin-6-yl)ethyl)piperazine-2,5-diones (Product 25).

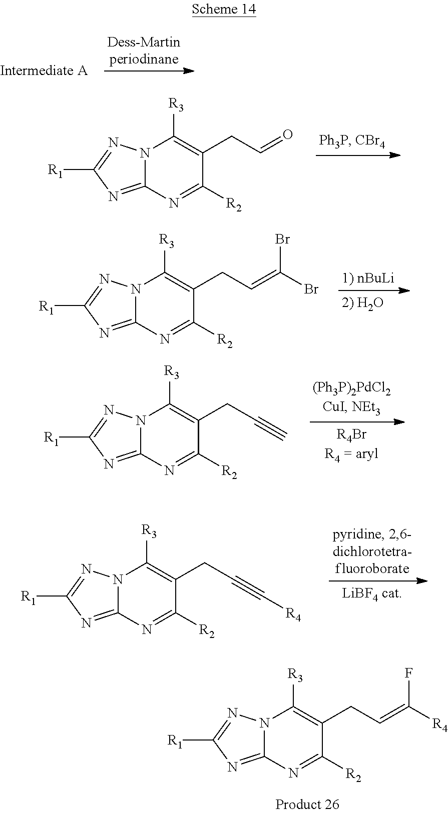

### General synthesis of 6-(3-fluoro-3-arylallyl)-[1,2,4]triazolo[1,5-a]pyrimidines (Product 26)

Intermediate A is reacted with Dess-Martin periodinane to give the corresponding aldehyde, which is then converted to the corresponding dibromoalkene and then to alkyne in a two-step Corey-Fuchs reaction. Sonogashira coupling with a suitably-substituted arylbromide R₄Br affords the corresponding 6-(3-arylprop-2-yn-1-yl)-[1,2,4]triazolo[1,5-a]pyrimidine, which is then converted into the corresponding 6-(3-fluoro-3-arylallyl)-[1,2,4]triazolo[1,5-a]pyrimidines (Product 26) by reaction with pyridine, 2,6-dichlorotetrafluoroborate and catalytic LiBF₄.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 1H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from 0, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (−I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 2012, 20(18):5658-5667; William J S et al., Journal of Labelled Compounds and Radiopharmaceuticals, 2010, 53(11-12):635-644; Modvig A et al., J Org Chem, 2014, 79:5861-5868. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). In particular, the compounds of formula (I) can be used in the monotherapeutic treatment or prevention of cancer (i.e., without administering any other anticancer agents until the treatment with the compound(s) of formula (I) is terminated). Accordingly, the invention also relates to a compound of formula (I) or a pharmaceutically acceptable salt thereof, or a pharmaceutical composition comprising any of the aforementioned entities in combination with a pharmaceutically acceptable excipient, for use in the monotherapeutic treatment or prevention of cancer.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents, including patent applications and scientific literature, are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures:

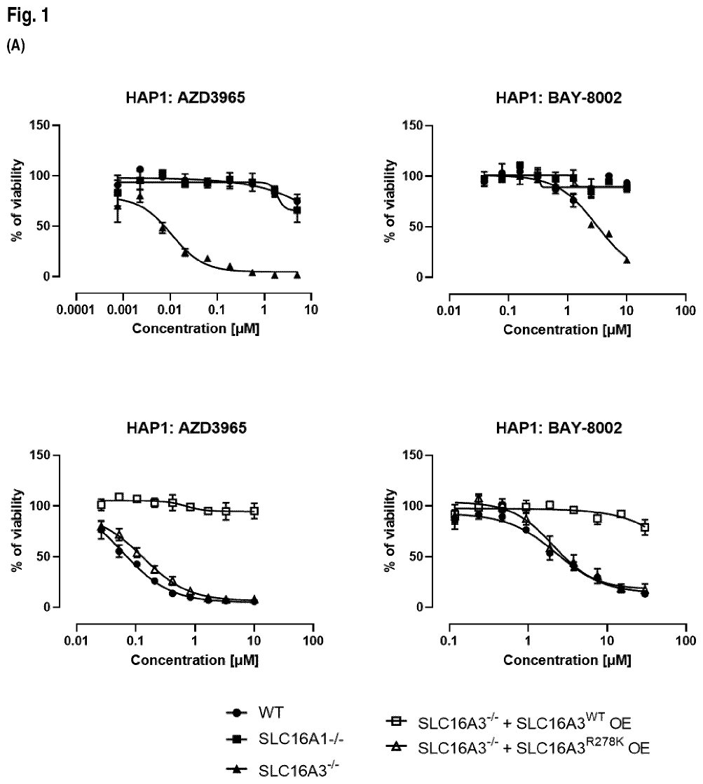

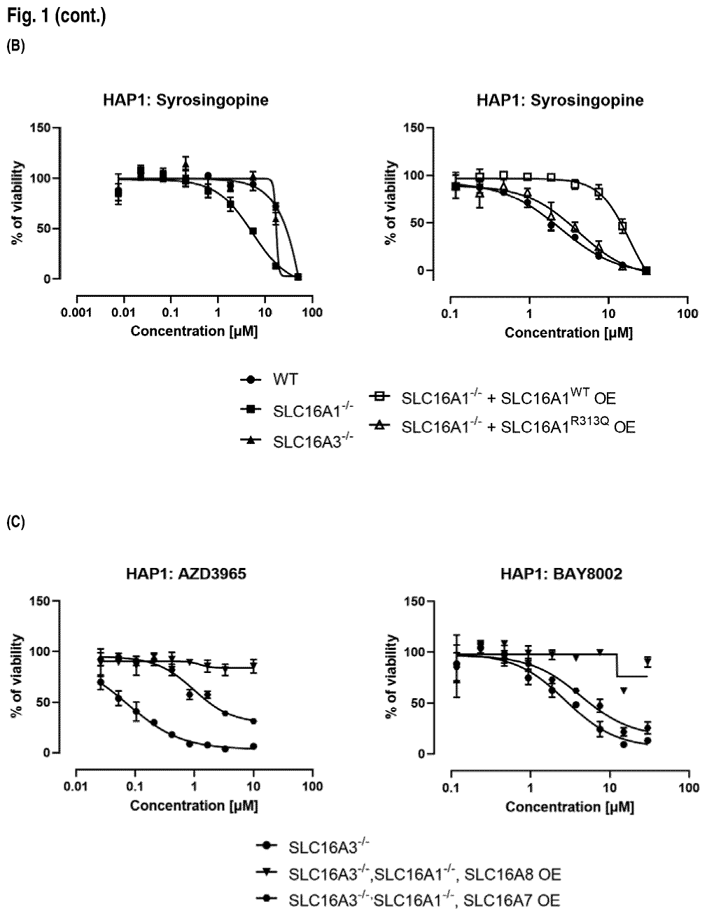

FIG. 1: HAP1 assay system. HAP1 cells are sensitive to SLC16A1 (A) inhibition upon SLC16A3 knock-out (KO), and this effect is rescued by re-introduction of wt SLC16A3 cDNA. Similarly, HAP1 SLC16A1 KO cells are sensitive to SLC16A3 inhibition (B). This effect can be disrupted by overexpression of SLC16A7 or SLC16A8 (C), which results in dependency on newly introduced SLC. See Example 1.

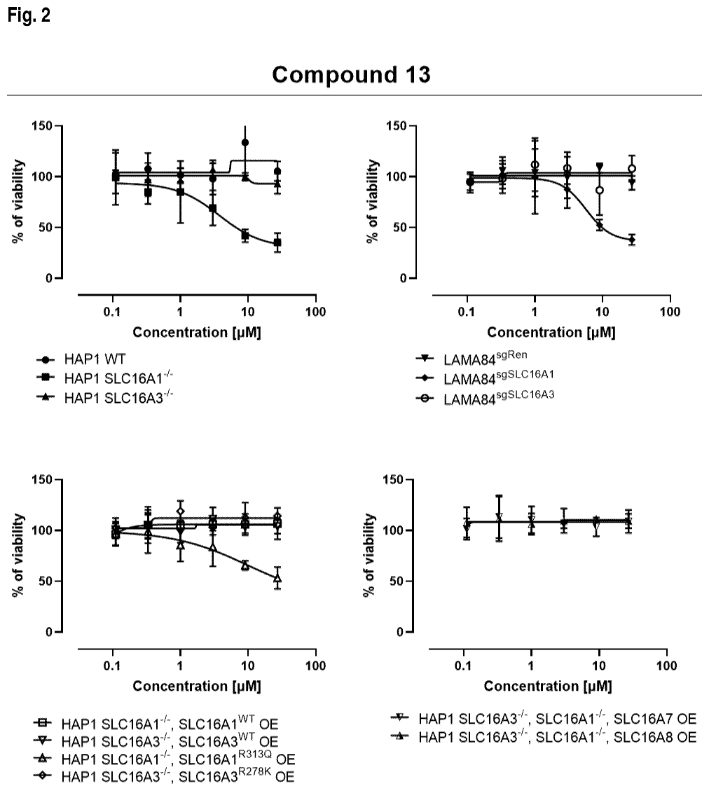

FIG. 2: Selectivity counter screen. Presented compounds selectively kill only cells dependent on SLC16A3 in HAP1 and LAMA84 cell lines. The killing effect is rescued by re-introduction of SLC16A1^{WT} cDNA but not by SLC16A1^{R313Q} cDNA, demonstrating that the effect is dependent on the transport function of the SLC. Compound 13 is given as an illustrative example.

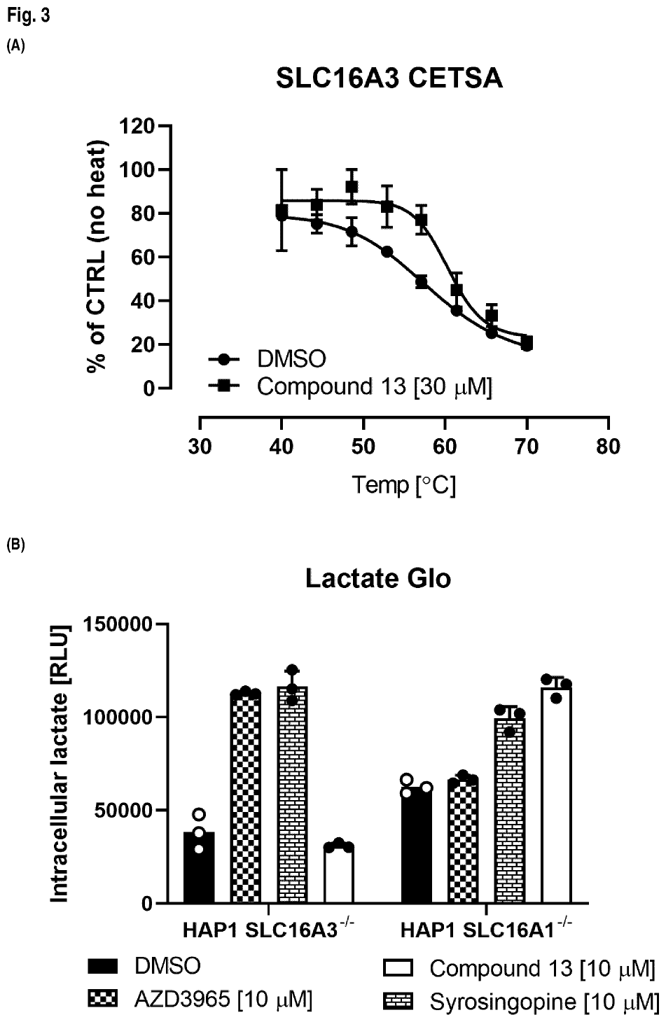

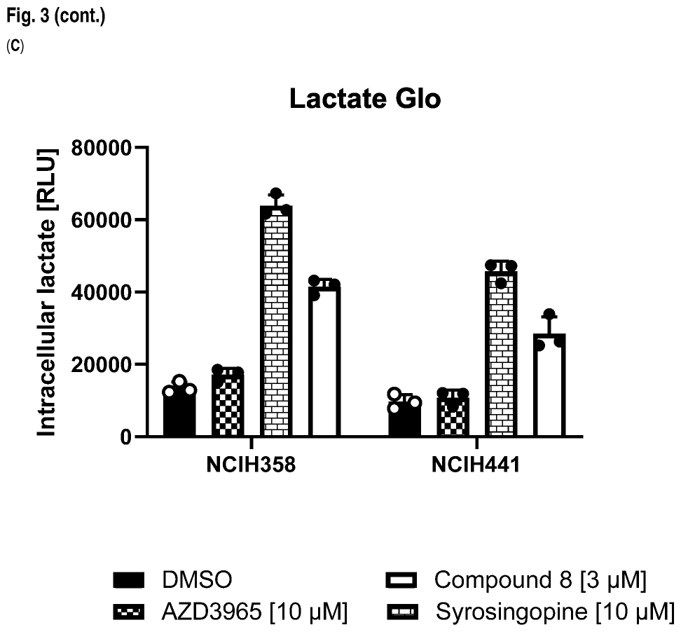

FIG. 3: Secondary assays confirmed SLC16A3 inhibition. Binding of the representative compound (Compound 13) was confirmed with CETSA (A). Incubation of SLC16A1^{-/-} cells with Compound 13 for 6 hours resulted in lactate accumulation comparable to control (syrosingopine), while no accumulation was observed in SLC16A3^{-/-} cells, confirming selectivity (B). Incubation of 2 lung cancer cell lines known for high expression of SLC16A3 with compound 8 for 6 hours resulted in lactate accumulation, validating the findings in HAP1 KO system in additional models (C). See Example 1.

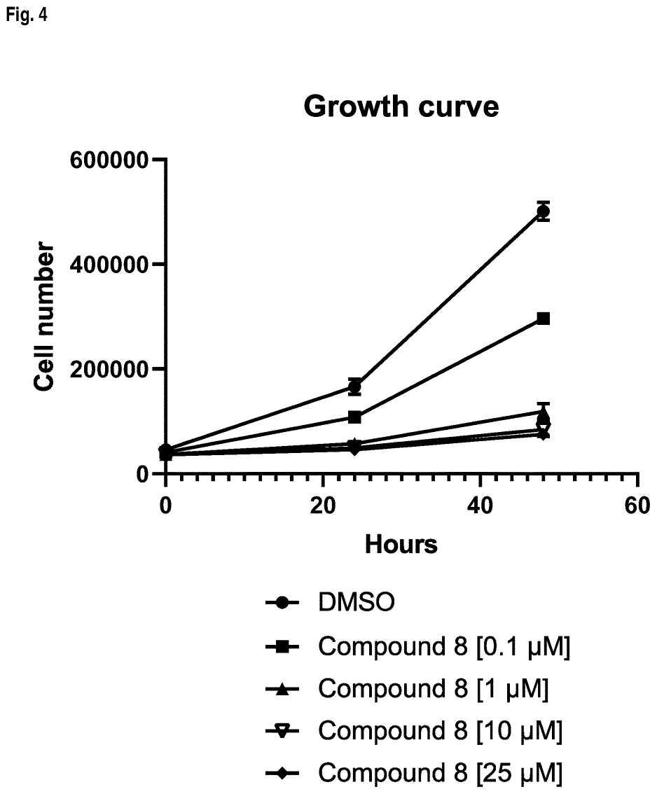

FIG. 4: Growth reducing effect of the SLC16A3 inhibitors. Treatment of HAP1 SLC16A1^{-/-} cells with different concentrations of SLC16A3 inhibitors (using compound 8 as a representative example) confirmed the growth reducing effect.

The invention will now be described by reference to the following examples which are merely illustrative and are not to be construed as a limitation of the scope of the present invention.

### EXAMPLES

### Example 1: Identification of Selective SLC16A3 Inhibitors

### Materials and Methods

### Cell Culture

HAP1, HAP1 SLC16A1^{-/-} and HAP1 SLC16A3^{-/-} were obtained from Haplogen. All HAP1 cell lines were grown in IMDM supplemented with 10% FBS and 1% penicillin-streptomycin. LAMA84^{sgRen}, LAMA84^{sgSLC16A1} and LAMA84^{sgSLC16A3} were generated previously (Pemovska et al., 2020, submitted to Nature Communications) and were grown in RPMI supplemented with 10% FBS and 1% penicillin-streptomycin. HEK293T cells were obtained from ATCC and cultured in DMEM supplemented with 10% FBS and 1% penicillin-streptomycin. NCIH441 and NCIH358 were obtained from laboratory of Georg Winter (CeMM Research Center for Molecular Medicine, Vienna, Austria) and cultured in RPMI supplemented with 10% FBS and 1% penicillin-streptomycin.

### Plasmids and Generation of Stable Cell Lines

Codon optimized cDNAs encoding SLC16A1, SLC16A3, SLC16A7 and SLC16A8 in pDONR221 were obtained from RESOLUTE consortium.

HIBIT tagging for CETSA experiments was performed on N-termini of SLCs. HIBIT tag and mutations in SLC16A1 and SLC16A3 (R313Q or R278K) were introduced in pDONR vectors using site-directed mutagenesis (NEB). cDNAs were then transferred into LEgwSHIB (pRRL-EF1a-gwSH-IRES-BlastR) or LEgwSTOPIB (pRRL-gwSTOP-IRES-BlastR) vectors (Bigenzahn J W et al., Science, 2018, 8210(November):eaap8210) using gateway cloning LR reaction (Invitrogen). For KO of SLC16A1, gRNA (CACCGACAGACGTATAGTTGCTGTA) was cloned into LentiCRISPRv3. All sequences were confirmed by Sanger sequencing.

Lentivirus was produced in HEK293T cells transiently co-transfected with psPAX2, pMD2.G and corresponding expression vector. 12 hours after transfection media was changed for collection of lentiviruses. After another 48-72 hours, media containing lentivirus was collected, filtered through 0.2 μm filter and either mixed with 5 μg/ml Polyberene (Hexadimethrine bromide, Sigma) and added to target cells, or stored at −80° C. 24 hours after transduction, virus containing medium was removed and replaced with fresh medium, and in additional 24 hours respective selection antibiotics (puromycin or blasticidin) were added to derive stably expressing cell lines.

### Chemical Screening

Compounds were printed to individual wells of 384 well plates (Corning) with Echo acoustic liquid dispenser (Labcyte). HAP1 cells were seeded using a dispenser (Thermo Fischer Scientific) in density 1,000 cells per well in 50 μl. As controls, DMSO or Bortezomib treatments were used. Viability of cells was assessed after 72 hours with CellTiter Glo (Promega) and % of controls were calculated based on RLU values in DMSO (100%) and Bortezomib (0%) wells. First screening was performed in 10 μM final concentration. Second screening was performed in technical duplicates, in 4 point dose response setting, with concentrations ranging from 13-0.5 μM. Third screening was performed in technical triplicates in 6 point dose response setting, with concentrations ranging from 27-0.11 μM. LAMA84 cells were used in density 3,000 cells per well. AZD3965, BAY-8002, syrosingopine and compounds obtained from third party vendors were tested in 96 well plates, with 2,500 cells per well with manual seeding and treatment. Compound efficacy was determined either by comparing % of control, or by IC₅₀ estimation based on dose response curves (calculated with R package drc, or with GraphPad Prism).

### Cellular Thermal Shift Assay

HEK293T cells with stable expression of HIBIT-tagged SLC16A3 were lysed as described in Hashimoto M et al., ACS Chemical Biology, 2018, 13(6):1480-86. Lysates were then centrifugated at 14,000 RPM, 20 min at 4° C. Supernatant were next incubated with tested compounds or DMSO on ice for 1 hour. After that samples were aliquoted to PCR strips, heated in gradient thermocycler for 6 min and let cool down at room temperature (RT) for 3 min. Samples were then centrifugated at 14,000 RPM, 40 min at 4° C. and protein abundance in supernatants was determined using HIBIT lytic detection system (Promega) in 384 well plate. Reagents for HIBIT detection were diluted in lysis buffer (Hashimoto M et al., loc. cit.) instead of buffer provided with kit. All measurements were done in technical triplicate.

### Lactate Glo

Protocol for lactate glo was modified based on Benjamin D et al., Cell Reports, 2018, 25(11):3047-3058.e4. Cells were seeded in 96 well plate in density 30,000 cells per well. Next day cells were treated with tested compounds, AZD3965 and syrosingopine (all in final concentration of 10 μM) or DMSO. After 6 hours of incubation at 37° C., cells were washed twice with PBS, lysed with 0.2N HCl, neutralized with 1M Tris-base (Sigma) and incubated for 1 hour at RT with lactate glo reagents (Promega). Lactate accumulation was then estimated based on luminescence signal.

### Growth Curve

Cells were seeded in 6 well plate in density of 15,000 cells per well in the presence of different concentrations of tested compounds in DMSO, or matching DMSO concentrations. Cells were washed with PBS, harvested with trypsin and counted with CASY counter (Roche) 24 h, 48 h and 72 h after the seeding. Cell number obtained at 24 h was set as 0, for each time point and concentration two wells were harvested and counted.

### Results

In order to set up a screening system for SLC16A3 modulators, the inventors took advantage of known genetic interaction between SLC16A1 and SLC16A3 (Girardi E et al., BioRxiv, 2020, doi:10.1101/2020.08.31.275818): HAP1 cells express both SLC16A1 and SLC16A3, and inhibition of either SLC16A1 or SLC16A3 is affecting viability minimally. However, the inhibition of SLC16A1 in HAP1 SLC16A3^{-/-} cells leads to severe reduction in fitness. This phenomenon can be also explored pharmacologically: treatment with AZD3965 or BAY-8002 reduced viability only in SLC16A3^{-/-} (see FIG. 1A), while the viability of HAP1 WT or HAP1 SLC16A1^{-/-} cells was not affected. Moreover, this effect was rescued when SLC16A3^{WT} cDNA was reintroduced into SLC16A3^{-/-} cells, but not when transport deficient SLC16A3^{R278K} cDNA was used, indicating that the observed effect depends on the transporter function of SLC16A3 (Sasaki S et al., PLoS ONE, 2013, 8(7):3-9). Similarly, syrosingopine decreased viability of SLC16A1^{-/-} cells compared to WT and SLC16A3^{-/-}, and this effect was rescued by SLC16A1^{WT} cDNA, but not SLC16A1^{R313Q} cDNA re-expression (see FIG. 1B) (van Hasselt P M et al., New England Journal of Medicine, 2014, 371(20):1900-1907). Thus, SLC16A1^{-/-} cells are dependent on SLC16A3 and vice versa.

Endogenously, HAP1 cells express none, or very low levels of SLC16A7 and SLC16A8 (Brockmann M et al., Nature, 2017, 546(7657):307-11), and thus the inventors hypothesized that exogenous expression of SLC16A7 or SLC16A8 will disrupt synthetic lethality between SLC16A1 and SLC16A3. To test this, the inventors overexpressed SLC16A7 or SLC16A8 in HAP1 SLC16A3^{-/-} cells and knocked out SLC16A1 using CRISPR/Cas9. They found that cells remain viable, indicating that the exogenous expression of SLC16A7 or SLC16A8 can rescue synthetic lethality. Moreover, upon knock-out (KO) of SLC16A1, cells should be dependent on exogenously expressed SLC16A7 or SLC16A8. To test this further, the inventors took advantage of the promiscuity of SLC16A1 inhibitors, which were reported to inhibit also SLC16A7, even though with lower potency (Quanz M et al., Molecular Cancer Therapeutics, 2018, 17(11):2285-96). They found that HAP1 dependent on SLC16A7 can be killed by AZD3965 and BAY-8002, indicating that the newly created cells lines are dependent on SLC16A7 or SLC16A8, respectively (see FIG. 1C).

To identify modulators of SLC16A3, the inventors performed a chemical screening using HAP1 SLC16A1^{-/-} cells exploiting their dependency on SLC16A3 and the expected reduction in their viability resulting from SLC16A3 inhibition. They obtained >1,400 compounds which reduced viability of cells to <50% compared to controls, which were tested further.

Next, the inventors screened previously selected compounds in HAP1 WT, HAP1 SLC16A1^{-/-} and HAP1 SLC16A3^{-/-} cell lines in 4 different concentration (13-05 μM, 3-fold dilution). They identified compounds which were toxic only to SLC16A1^{-/-} cells, indicating that the target of these compounds has a genetic interaction with SLC16A1. Interestingly, among these hits were 7 compounds sharing a common triazolopyrimidine scaffold (Table 1, Compounds 11-17).

To get further insights into the selectivity and mode of action of these hits, the inventors created a selection of compounds with similar structure to their hits from the whole library and tested those further. They used a broader panel of cell lines, which included HAP1 WT, HAP1 SLC16A1^{-/-}, HAP1 SLC16A3^{-/-}, LAMA84^{sgRen}, LAMA84^{sgSLC16A1} and LAMA84^{sgSLC16A3} (see FIG. 2 with compound 13 as a representative example). Furthermore, to probe selectivity, they included HAP1 cell lines with dependency on SLC16A7 and SLC16A8, and to probe transport dependency, they included also HAP1 SLC16A1^{-/-}, SLC16A1^{WT} OE; HAP1 SLC16A1^{-/-}, SLC16A1^{R313Q} OE; HAP1 SLC16A3^{-/-}, SLC16A3^{WT} OE and HAP1.

The inventors were able to confirm that a range of tested compounds killed HAP1 SLC16A1^{-/-}; LAMA84^{sgSLC16A1} but not HAP1 WT, HAP1 SLC16A3^{-/-}, LAMA84^{sgRen}, LAMA84^{sgSLC16A3} or HAP1 with dependency on either SLC16A7 or SLC16A8, indicating specificity to SLC16A3 over SLC16A1, SLC16A7 and SLC16A8. At the same time, killing was rescued in HAP1 SLC16A1′H with reintroduced SLC16A1^{WT} but not transport-deficient SLC16A1^{R313Q}, indicating that compounds are affecting the lactate transport in SLC16A3 dependent cell lines.

The observed effects of various tested compounds on HAP1 SLC16A1^{-/-}, HAP1 SLC16A3^{-/-} and HAP1 WT cells are summarized in the following Table 1:

| | | HAP1 | HAP1 | HAP1 | |
|---|---|---|---|---|---|
| | | SLC16A1^{-/-} | SLC16A1^{-/-} | SLC16A31^{-/-} | HAP1 WT |
| Compound | Structure | % of viability | IC₅₀ [μM] | IC₅₀ [μM] | IC₅₀ [μM] |
| 1 | 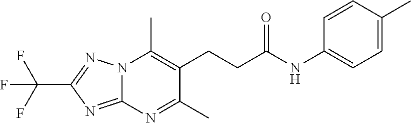 [SMILES: Cc1ccc(NC(=O)CCc2c(C)nc3nc(C(F)(F)F)nn3c2C)cc1] | — | 3.32 | >30 | >30 |
| 2 | 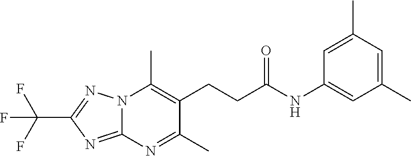 [SMILES: Cc1cc(C)cc(NC(=O)CCc2c(C)nc3nc(C(F)(F)F)nn3c2C)c1] | — | 4.13 | >30 | >30 |
| 3 | 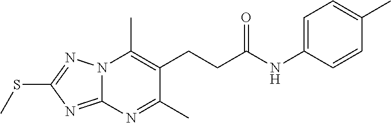 | — | 2.34 | >30 | >30 |
| 4 | 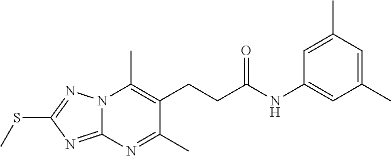 | — | 1.83 | >30 | >30 |
| 5 | 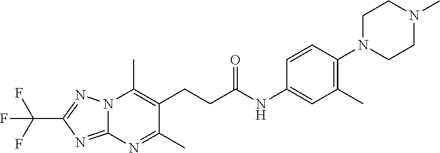 [SMILES: Cc1cc(NC(=O)CCc2c(C)nc3nc(C(F)(F)F)nn3c2C)ccc1N1CCN(C)CC1] | — | 5.87 | >30 | >30 |
| 6 | 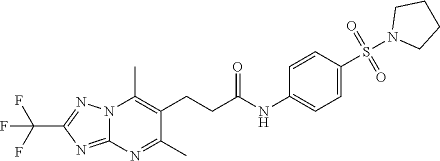 [SMILES: Cc1nc2nc(C(F)(F)F)nn2c(C)c1CCC(=O)Nc1ccc(S(=O)(=O)N2CCCC2)cc1] | — | 0.41 | >30 | >30 |
| 7 | 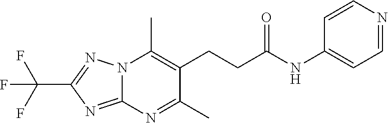 [SMILES: Cc1nc2nc(C(F)(F)F)nn2c(C)c1CCC(=O)Nc1ccncc1] | — | 11.17 | >30 | >30 |
| 8 | 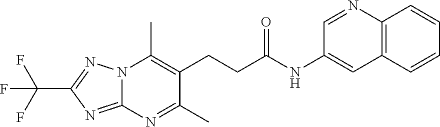 [SMILES: Cc1nc2nc(C(F)(F)F)nn2c(C)c1CCC(=O)Nc1cnc2ccccc2c1] | — | 0.25 | >30 | >30 |
| 9 | 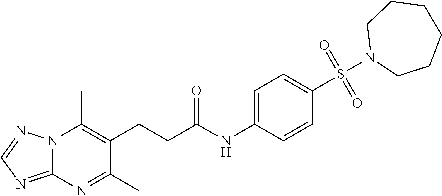 [SMILES: Cc1nc2ncnn2c(C)c1CCC(=O)Nc1ccc(S(=O)(=O)N2CCCCCC2)cc1] | — | 1.98 | >30 | >30 |
| 10 | 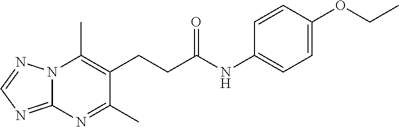 [SMILES: CCOc1ccc(NC(=O)CCc2c(C)nc3ncnn3c2C)cc1] | 70.82 | 7.07 | >30 | >30 |
| 11 | 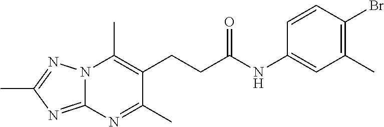 | 33.73 | — | — | — |
| 12 | 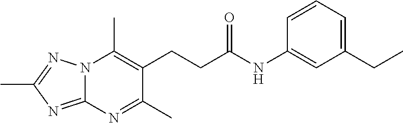 [SMILES: CCc1cccc(NC(=O)CCc2c(C)nc3nc(C)nn3c2C)c1] | 36.43 | — | — | — |
| 13 | 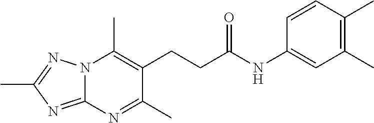 | 37.01 | — | — | — |
| 14 | 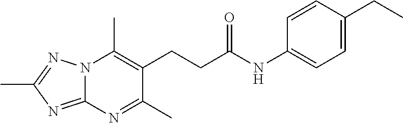 [SMILES: CCc1ccc(NC(=O)CCc2c(C)nc3nc(C)nn3c2C)cc1] | 37.55 | — | — | — |
| 15 | 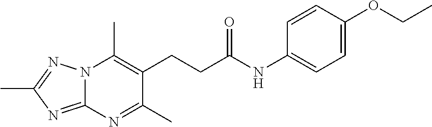 [SMILES: CCOc1ccc(NC(=O)CCc2c(C)nc3nc(C)nn3c2C)cc1] | 38.46 | — | — | — |
| 16 | 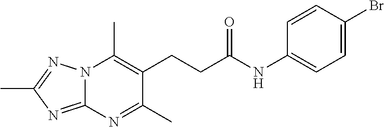 [SMILES: Cc1nc2nc(C)c(CCC(=O)Nc3ccc(Br)cc3)c(C)n2n1] | 38.72 | — | — | — |
| 17 | 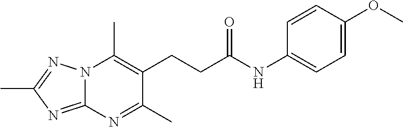 | 44.42 | — | — | — |
| 18 | 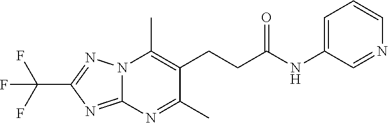 [SMILES: Cc1nc2nc(C(F)(F)F)nn2c(C)c1CCC(=O)Nc1cccnc1] | — | 8.41 | >30 | >30 |
| 19 | 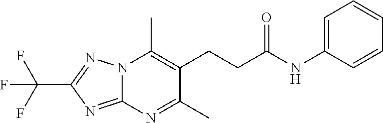 [SMILES: Cc1nc2nc(C(F)(F)F)nn2c(C)c1CCC(=O)Nc1ccccc1] | — | 8.94 | >30 | >30 |

Table 1: Results of synthetic lethality guided chemical screening assay. HAP1 WT, HAP1 SLC16A1^{-/-} or HAP1 SLC16A3^{-/-} cell lines were treated with presented compounds for 72 hours and viability was determined by Cell titer glo assay (Promega). Data are presented either as a percentage of viability in HAP1 SLC16A1^{-/-} cells compared to controls (DMSO=100% and Bortezomib=0%) upon single dose treatment (10 μM) or as IC₅₀ values obtained from dose response curves.

It has thus been demonstrated that a range of exemplary compounds according to the present invention, including in particular compounds 1 to 19, exhibit a potent inhibitory effect on SLC16A3 (as reflected by their viability reduction or IC₅₀ values on HAP1 SLC16A1^{-/-} cells). At the same time, the tested compounds exhibit no or insignificant inhibitory activity on SLC16A1 (as reflected by their IC₅₀ values on HAP1 SLC16A3^{-/-} cells), which indicates that these inhibitors are advantageously selective for SLC16A3 over SLC16A1.

To further characterize the identified hit compounds, the inventors picked compounds 8 and 13 as representative examples. First, they performed Cellular Thermal Shift Assay (CETSA) on SLC16A3 with HEK293T cells stably expressing HIBIT-tagged SLC16A3 employing split-nano luciferase as a readout. Treatment with compound 13 showed a shift in melting temperature of SLC16A3 indicating that this compound is binding SLC16A3 (see FIG. 3A). Second, to confirm a direct inhibition of lactate export, the inventors measured lactate accumulation in HAP1 SLC16A1^{-/-} and HAP1 SLC16A3^{-/-} cells with Lactate Glo assay upon treatment with compound 13. While this compound did not affect lactate accumulation in SLC16A3^{-/-} cells, significant lactate accumulation was observed in SLC16A1^{-/-} cells, confirming that compound 13 is a specific inhibitor of SLC16A3 compared to SLC16A1 (see FIG. 3B). Third, to test the compounds in in vitro models of lung cancer, the inventors measured lactate accumulation in NCIH358 and NCIH441 cells, known to those skilled in art for high expression of SLC16A3 and low expression of SLC16A1 and SLC16A7, upon treatment with compound 8 using Lactate Glo assay. The results thus obtained confirmed the lactate accumulation in both cell lines upon the treatment with compound 8 (see FIG. 3C). Fourth, to test the effect of compounds on cell proliferation, the inventors assessed the growth rate of HAP1 SLC16A1^{-/-} upon treatment with compound 8. The treatment slowed down the proliferation in a dose-dependent manner (see FIG. 4). These findings further confirm the advantageous therapeutic activity of the compounds provided herein.

1. A compound of formula (I) 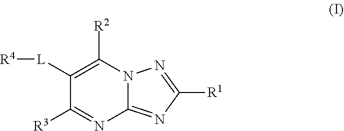
or a pharmaceutically acceptable salt thereof, wherein:
R¹ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—;
R² and R³ are each independently selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—;
R⁴ is aryl or heteroaryl, wherein said aryl or said heteroaryl is optionally substituted with one or more groups R⁴¹, and further wherein said heteroaryl is not 1,2,4-triazolyl;
each R⁴¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), -L^{C1}-R^{C1}, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}, and further wherein one or more —CH₂— units comprised in the C₀₋₃ alkylene moiety of said —(C₀₋₃ alkylene)-carbocyclyl or of said —(C₀₋₃ alkylene)-heterocyclyl are each optionally replaced by a group independently selected from —O—, —NH—, —N(C₁₋₅ alkyl)-, —CO—, —S—, —SO—, and —SO₂—;
L is C₂₋₈ alkylene, wherein one —CH₂— unit in said alkylene is replaced by a group —R^{L1}—, wherein said alkylene is optionally substituted with one or more groups R^{L2}, and optionally wherein one or more —CH₂— units in said alkylene are each replaced by a group independently selected from —O—, —NH—, —N(C₁₋₅ alkyl)-, —CO—, —S—, —SO— and —SO₂—;
R^{L1} is selected from —CO—N(R^{N})—, —N(R^{N})—CO—, —CO—O—, —O—CO—, —SO₂—N(R^{N})—, —N(R^{N})—SO₂—, —CO—O—N(R^{N})—, —N(R^{N})—CO—O—, —CS—N(R^{N})—, —N(R^{N})—CS—, —N(R^{N})—CO—N(R^{N})—, —C(═N(R^{N}))—N(R^{N})—, —N(R^{N})—C(═N(R^{N}))—, —C(—CF₃)—N(R^{N})—, —N(R^{N})—C(—CF₃)—, —O—P(═O)(C₁₋₅ alkyl)-N(R^{N})—, —N(R^{N})—P(═O)(C₁₋₅ alkyl)-O—, —CH═CH—, —CF═CH—, —CH═CF—, imidazoldiyl, thiazoldiyl, triazoldiyl, oxadiazoldiyl, tetrazoldiyl, diketopiperazindiyl, -oxetandiyl-N(R^{N})—, and —N(R^{N})-oxetandiyl-;
each R^{N} is independently hydrogen or C₁₋₅ alkyl, wherein said alkyl is optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each R^{L2} is independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), -L^{C1}-R^{C1}, —(C₀₋₃ alkylene)-cycloalkyl, and —(C₀₋₃ alkylene)-heterocycloalkyl;
each L^{C1} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{C1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
for use in the treatment or prevention of an SLC16A3-associated disease/disorder.

2. The compound for use according to claim 1, wherein R¹ is selected from hydrogen, C₁₋₅ alkyl, —O(C₁₋₅ alkyl), —S(C₁₋₅ alkyl), halogen, C₁₋₅ haloalkyl, —CN, carbocyclyl, and heterocyclyl, wherein said carbocyclyl and said heterocyclyl are each optionally substituted with one or more R^{Cyc}; preferably wherein R¹ is selected from hydrogen, C₁₋₅ alkyl, —O(C₁₋₅ alkyl), —S(C₁₋₅ alkyl) and C₁₋₅ haloalkyl.

3. The compound for use according to claim 1 or 2, wherein R² and R³ are each independently selected from hydrogen, C₁₋₅ alkyl, —O(C₁₋₅ alkyl), —S(C₁₋₅ alkyl), halogen, C₁₋₅ haloalkyl, —CN, carbocyclyl, and heterocyclyl, wherein said carbocyclyl and said heterocyclyl are each optionally substituted with one or more R^{Cyc}; preferably wherein R² and R³ are each independently C₁₋₅ alkyl.

4. The compound for use according to any one of claims 1 to 3, wherein R⁴ is phenyl or monocyclic heteroaryl, wherein said phenyl or said monocyclic heteroaryl is optionally substituted with one or more R⁴¹, and wherein said monocyclic heteroaryl is not 1,2,4-triazolyl; preferably wherein R⁴ is phenyl or pyridinyl, wherein said phenyl or said pyridinyl is optionally substituted with one or more R⁴¹.

5. The compound for use according to any one of claims 1 to 4, wherein L is C₂₋₈ alkylene, wherein one —CH₂— unit in said alkylene is replaced by a group —CO—N(R^{N})—, wherein said alkylene is optionally substituted with one or more groups R^{L2}.

6. The compound for use according to any one of claims 1 to 5, wherein L is a group —(C₁₋₄ alkylene)-CO—N(R^{N})—, wherein said alkylene is optionally substituted with one or more R^{L2}, and wherein said group is attached via its —CO—N(R^{N})— moiety to R⁴; preferably L is a group —CH₂CH₂—CO—N(R^{N})— which is attached via its —CO—N(R^{N})— moiety to R⁴.

7. The compound for use according to any one of claims 1 to 6, wherein each R^{N} is independently hydrogen or C₁₋₅ alkyl.

8. The compound for use according to claim 1, wherein said compound is selected from: 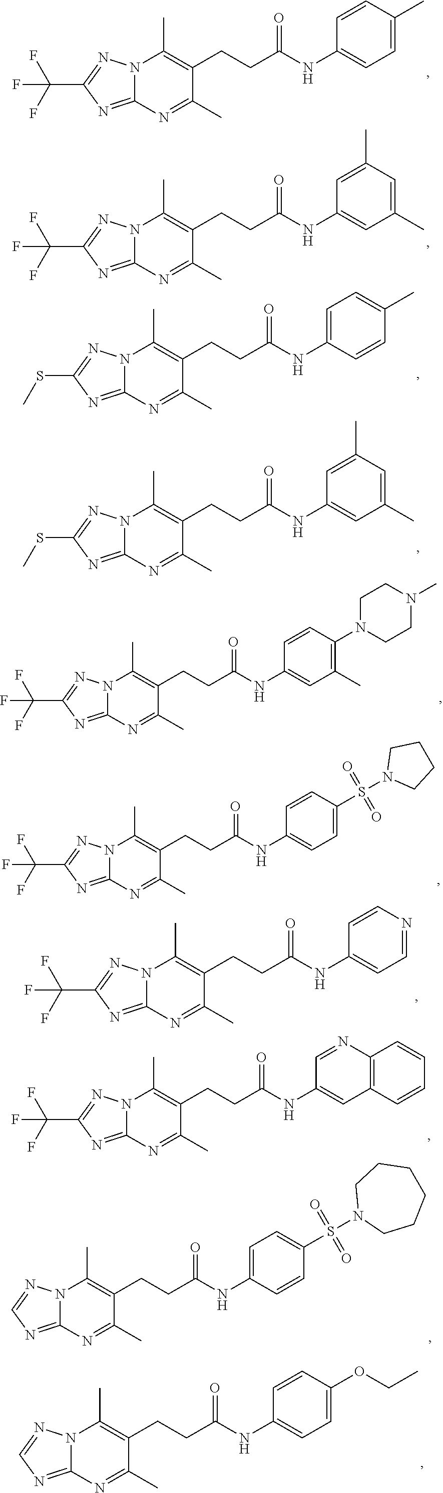 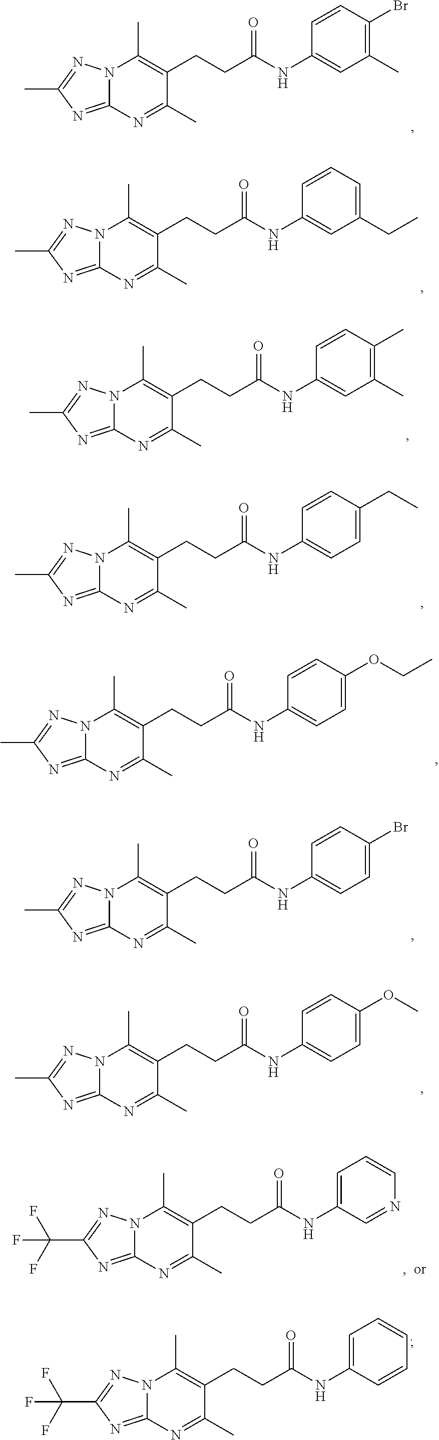
or a pharmaceutically acceptable salt of any one of the aforementioned compounds.

9. A pharmaceutical composition for use in the treatment or prevention of an SLC16A3-associated disease/disorder, wherein the pharmaceutical composition comprises a compound as defined in any one of claims 1 to 8 and a pharmaceutically acceptable excipient.

10. The compound for use according to any one of claims 1 to 8 or the pharmaceutical composition for use according to claim 9, wherein said compound or said pharmaceutical composition is for use in the treatment or prevention of cancer.

11. The compound for use according to claim 10 or the pharmaceutical composition for use according to claim 10, wherein said cancer is selected from lung cancer, cervical cancer, colorectal cancer, colon cancer, rectal cancer, glioblastoma, gastric cancer, ovarian cancer, head and neck squamous cell carcinoma, oral squamous cell carcinoma, breast cancer, prostate cancer, bladder cancer, liver cancer, renal cancer, thyroid cancer, pancreatic cancer, bone cancer, leukemia, lymphoma, melanoma, endometrial cancer, uterine sarcoma, and multiple myeloma.

12. The compound for use according to any one of claims 1 to 8 or the pharmaceutical composition for use according to claim 9, wherein said compound or said pharmaceutical composition is for use in the treatment or prevention of an inflammatory disorder, a cardiovascular disorder, a fibrotic disorder, or Alzheimer's disease.

13. In vitro use of a compound as defined in any one of claims 1 to 8 as an SLC16A3 inhibitor.
